(19) United States
(12) Patent Application Publication
(10) Pub. No.: US 2024/0315949 A1
(43) Pub. Date: Sep. 26, 2024

(54) COATING COMPOSITIONS AND APPLICATIONS THEREOF

(71) Applicant: AESCULATECH, INC., LOS ANGELES, CA (US)

(72) Inventors: Roby Menefee, Los Angeles, CA (US); Thomas Saal, Los Angeles, CA (US); Yulin Chen, Irvine, CA (US); Adam Ung, Alhambra, CA (US)

(21) Appl. No.: 18/610,507

(22) Filed: Mar. 20, 2024

Related U.S. Application Data
(60) Provisional application No. 63/453,669, filed on Mar. 21, 2023.

(51) Int. Cl. A61K 8/88; A61K 8/34; A61K 8/81; A61K 8/85; A61K 8/9789; A61Q 3/02; A61Q 3/04

(52) U.S. Cl. CPC A61K 8/88; A61K 8/34; A61K 8/8152; A61K 8/85; A61K 8/9789; A61Q 3/02; A61Q 3/04; A61K 2800/594

(57) ABSTRACT

Described herein are coating compositions that can be applied to a number of different substrates. The coating compositions can be used as decorative or protective layers across a range of applications and formulations such as, for example, cosmetics. In one aspect, the coating composition includes a solvent, one or more polymers, and optional additives for enhancing or modifying the properties of the coating composition such as, for example, mechanical properties, rheological properties, appearance, and environmental sensitivity. When applied to the substrate, the coating composition forms a polymer film as the solvent present in the coating composition evaporates. The coating compositions may be easily removed from the substrate with the use of a solvent or a removal solution that reacts or mechanically weakens the film.

### CROSS-REFERENCE TO RELATED APPLICATIONS

This application claims the benefit of and priority to co-pending U.S. Provisional Patent Application No. 63/453,669, filed on Mar. 21, 2023, the contents of which are incorporated by reference herein in their entireties.

### BACKGROUND

Coatings are used across a range of industries and consumer goods to protect and improve the appearance of surfaces. A surface might be composed of a wide range of materials and exposed to a similarly diverse range of environments. As a result, ingredients and compositions which provide optimal performance—characterized by durability, appearance, and versatility—are in demand. Advances in these categories are achieved both through the development of new film-forming polymers, but also in the novel combination of known materials to produce better behavior. Interest in safety, particularly for coatings used in consumer products, has further motivated the use of new materials that invite fewer health hazards.

### SUMMARY

Described herein are coating compositions that can be applied to a number of different substrates. The coating compositions can be used as decorative or protective layers across a range of applications and formulations such as, for example, cosmetics. In one aspect, the coating composition includes a solvent, one or more polymers, and optional additives for enhancing or modifying the properties of the coating composition such as, for example, mechanical properties, rheological properties, appearance, and environmental sensitivity. When applied to the substrate, the coating composition forms a polymer film as the solvent present in the coating composition evaporates. The coating compositions may be easily removed from the substrate with the use of a solvent or a removal solution that reacts or mechanically weakens the film.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below:

FIG. 1 shows a number of pigmented coating compositions as described applied to a ceramic tile.

FIG. 2 shows a pigmented coating composition as described applied to a ceramic tile with and without a protective cellulose ester coat.

FIG. 3 shows a pigmented coating composition being applied to a fingernail followed by a clear protective coat.

FIG. 4 shows a completed application of a red pigmented coating and clear top coat to the fingernails of a hand.

FIG. 5 shows a handful of possible colors which may be formed through the combination of the coatings described herein with a variety of pigments and filers. The coats are applied to fingernails in this emobodiment.

FIG. 6 shows the removal of the pigmented coating composition from the nails of a hand using a removal solution composed of ethanol, water, niacinamide, panthenol, and lavender essential oil.

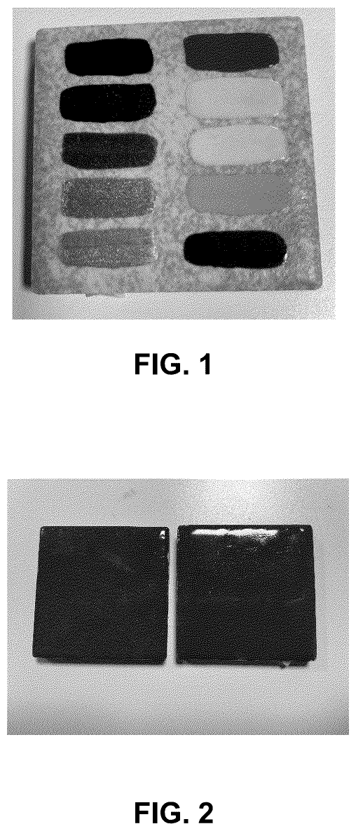

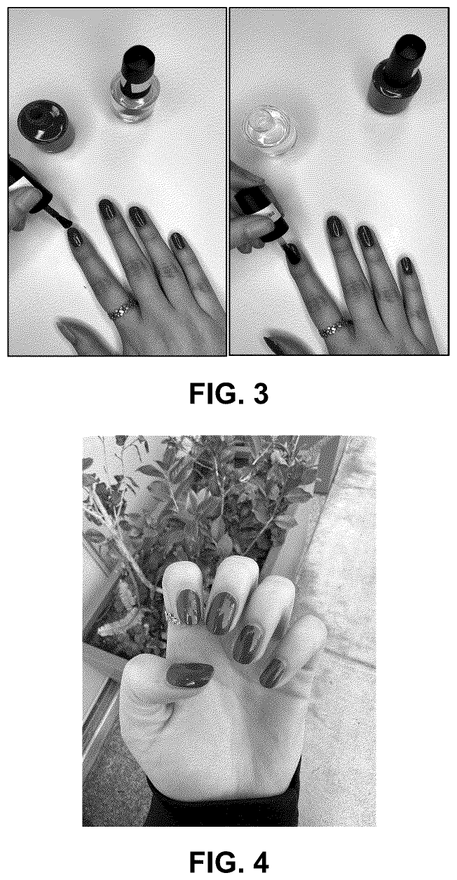

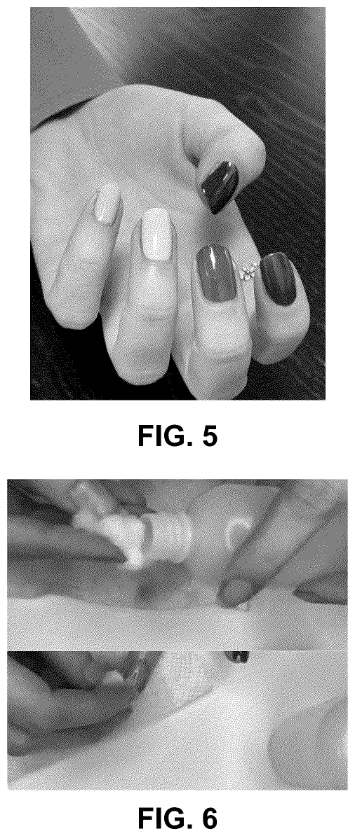

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an example, a numerical range of “about 1” to “about 5” should be interpreted to include not only the explicitly recited values of about 1 to about 5, but also to include individual values and sub-ranges within the indicated range. Thus, included in this numerical range are individual values such as 2, 3, and 4, the sub-ranges such as from 1-3, from 2-4, from 3-5, from about 1-about 3, from 1 to about 3, from about 1 to 3, etc., as well as 1, 2, 3, 4, and 5, individually. The same principle applies to ranges reciting only one numerical value as a minimum or maximum. The ranges should be interpreted as including endpoints (e.g., when a range of “from about 1 to 3” is recited, the range includes both of the endpoints 1 and 3 as well as the values in between). Furthermore, such an interpretation should apply regardless of the breadth or range of the characters being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed, that while specific reference to each various individual combination and permutation of these compounds may not be explicitly disclosed, each is specifically contemplated and described herein. For example, if a thermoresponsive polymer is disclosed and discussed, and a number of different additives are discussed, each and every combination of thermoresponsive polymer and additive that is possible is specifically contemplated unless specifically indicated to the contrary. For example, if a class of thermoresponsive polymers A, B, and C are disclosed, as well as a class of additives D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination A+D. Likewise, any subset or combination of these is also specifically contemplated and disclosed. Thus, for example, the sub-group of A+E, B+F, and C+E is specifically contemplated and should be considered from disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such composition is specifically contemplated and should be considered disclosed.

In the specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “an additive” includes mixtures of two or more additives and the like.

“Optional” or “optionally” means that the subsequently described event or circumstance can or cannot occur, and that the description includes instances where the event or circumstance occurs and instances where it does not.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

It should be noted that ratios, concentrations, amounts, and other numerical data can be expressed herein in a range format. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “alkyl group” is a branched or unbranched saturated hydrocarbon group of 1 to 10 carbon atoms, such as methyl, ethyl, n-propyl, isopropyl, n-butyl, isobutyl, t-butyl, pentyl, hexyl, heptyl, octyl, decyl, and the like.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, an acrylic acid residue in a copolymer used herein refers to one or more —CH₂CH(CO₂H)— units in the copolymer, regardless of whether acrylic was used to produce the copolymer.

As used herein, the term “polymer” may refer to a homo-polymer, a copolymer, a tri-polymer and other multi-polymer, or a mixture thereof.

As used herein, the term “vinyl polymer” includes all polymers derived from vinyl monomers which have a backbone chain comprised of covalently linked carbon atoms. Vinyl polymers may be homopolymers, copolymers with 2 or more constituent monomer groups, cross-linked, or mixed. Cross linked vinyl polymers may have backbones which are not exclusively covalently bonded carbon atoms, or backbone regions which are not exclusively covalently bonded carbon atoms.

As used herein, the term “lower critical solution temperature” (LCST) or “lower consolute temperature” is the critical temperature below which a thermoresponsive polymer is miscible for all compositions.

As used herein, the term “admixing” is defined as mixing two or more components together so that there is no chemical reaction or physical interaction. The term “admixing” also includes the chemical reaction or physical interaction between the two or more components.

As used herein, the term “subject” or “individual” as used herein includes mammals. Non-limiting examples of mammals include humans, dogs, cats, and mice, including transgenic and non-transgenic mice. The methods described herein can be useful in both human therapeutics, pre-clinical, and veterinary applications. In some embodiments, the subject is a mammal, and in some embodiments, the subject is human.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can include one or multiple documents and are meant to include future updates.

### Coating Compositions

Described herein are coating compositions that can be applied to a number of different substrates. The coating compositions can be used as decorative or protective layers across a range of applications and formulations such as, for example, cosmetics. In one aspect, the coating composition includes a solvent, one or more polymers, and optional additives for enhancing or modifying the properties of the coating composition such as, for example, mechanical properties, rheological properties, appearance, and environmental sensitivity.

### Copolymers

The coating compositions described herein include one or more polymers. In one aspect, the coating compositions include one or more copolymers. A copolymer is a polymer having two or more different types of monomers joined in the same polymer chain. In certain embodiments, a composition herein comprises a block copolymer. Block copolymers are made up of blocks of different polymerized monomers. In certain aspects, the topical composition herein comprises a random copolymer. Random copolymers are made up of repeating units that are dispersed irregularly along the linear chains. In certain aspects, the topical composition herein comprises cross-linked copolymers. Cross-linked copolymers are polymers that are linked to one another by covalent or ionic or hydrogen bonds.

In one aspect, one or more of the polymers in the coating composition is a vinyl copolymer. The vinyl copolymers have a carbon-carbon backbone. The monomers used to produce the copolymer can be ordered, randomly distributed, or some combination of ordered and random. This can take the form of block copolymer, random copolymer, or graft copolymer.

In certain aspects, the vinyl polymers described herein can be combined to create a composition having two or more different polymers. For example, vinyl polymers above can be combined to create block copolymers, random copolymers, and cross-linked copolymers.

In one aspect, the vinyl copolymers polymers contain alkyl acrylates and acrylamide. In other embodiments, the vinyl copolymers contain residues of (1) one or more alkyl acrylates and (2) acrylamide, methacrylamide, an alkyl acrylamide, a dialkyl acrylamide, an alkyl methacrylamide, a dialkyl methacrylamide, or any combination thereof. In another aspect, the vinyl copolymers include residues of an alkyl acrylate and an alkylacrylamide (e.g., methacrylamide). In another aspect, the vinyl copolymer includes a residue of an alkyl acrylate and n-alkyl alkylacrylamide (n-isopropylmethacrylamide).

The combination of different monomers can provide control over solubility, durability, and gloss. In one aspect, alkyl acrylates can improve the durability of glossy acrylamide polymers. For example, a homopolymer of butyl acrylate is rubbery and flexible when produced via free radical polymerization in THF. A homopolymer of n-isopropylacrylamide is glossy but brittle. By combining these two monomers, a high-gloss, flexible film can be attained. In other aspects, additional monomers can be included in the copolymer structure to provide further enhancement of appearance, chemical, or mechanical properties.

Additional monomers can improve the chemical properties of the polymers previously discussed. For example, acrylic acid can improve adhesion to surfaces. It also lowers the coating pH under the right conditions when water is present. This can improve coating adherence to a range of surfaces as well as provide unique removal behavior. For example, a removal solution that incorporates a base can react with the acrylic acid monomer to compromise the film and facilitate removal.

Additional monomers can improve the strength and durability of the copolymers present in the coating compositions. For example, polymethyl methacrylate is a transparent thermoplastic that possesses rigidity and chemical resistance. Incorporation of the methyl methacrylate monomer into the copolymers described herein can improve water resistance, surface hardness, and flexibility. Many acrylamide and n-alkyl acrylamides absorb water or interact with it to some degree. Methyl methacrylate can help limit the amount of these interactions in a coating film.

In one aspect, the alkyl acrylate is a C₁-C₁₈ alkyl acrylate. Examples of alkyl acrylates include, but are not limited to, methyl acrylate, ethyl acrylate, propyl acrylate, butyl acrylate, pentyl acrylate, hexyl acrylate, 2-ethylhexyl acrylate, styrene acrylate, heptyl acrylate, octyl acrylate, nonyl acrylate, decyl acrylate, undecyl acrylate, dodecyl acrylate, hexadecyl acrylate, stearyl acrylate, lauryl acrylate, t-butyl acrylate, isobutyl acrylate, isobornyl acrylate and any combination thereof.

In another aspect, the copolymer includes a residue of n-methyl acrylamide, n-ethylacrylamide, n-isopropylacrylamide, n-butylacrylamide, n-tertbutylacrylamide, n-methylmethacrylamide, n-ethyl methacrylamide, n-isopropyl methacrylamide, n-butyl methacrylamide, n-tertbutyl methacrylamide, and any combination thereof.

In other aspect, the copolymers include residues of monomers having additional functional groups such as a hydroxyl group to improve solubility or compatibility within the coating composition. One such example is hydroxyethyl acrylamide.

In certain aspect, the copolymer includes residues of an alkyl acrylate and alkyl acrylamide. Selected alkyl acrylamides offer improved gloss and hardness while selected alkyl acrylates provide improved plasticity and durability. The combination of these constituents further allows tuning of polymer solubility, a critical parameter for coating properties that dictates the volatile solvent used in formulation in addition to the compatibility of additional ingredients. In one aspect, the copolymer includes residues from n-isopropylacrylamide, butyl acrylate, and acrylic acid. In another aspect, the copolymer includes residues from n-isopropylmethacrylamide, butyl acrylate, and acrylic acid. In another aspect, the copolymer includes residues from n-isopropylmethacrylate and ethylhexyl acrylate. In another aspect, the copolymer comprises residues from n-isopropylmethacrylate, ethylhexyl acrylate, and acrylic acid.

In one aspect, the copolymer in the coating composition is a selected from one of the following: poly(butyl acrylate/acrylamide), poly(ethyl hexyl acrylate/acrylamide), poly(butyl acrylate/acrylic acid),), poly (butyl acrylate/n-isopropylacrylamide), poly (butyl acrylate/n-isopropylacrylamide/acrylamide), poly (butyl acrylate/n-isopropylacrylamide/acrylamide), poly(ethyl hexyl acrylate/n-isopropylacrylamide), poly(ethyl hexyl acrylate/n-isopropylacrylamide/acrylic acid), poly(ethyl hexyl acrylate/n-isopropylacrylamide/methyl methacrylate), poly(ethylhexyl acrylate/n-isopropylacrylamide/acrylic acid), poly(ethylhexyl acrylate/n-isopropylacrylamide/acrylic acid/methyl methacrylate), poly (butyl acrylate/n-butylacrylamide), poly (butyl acrylate/n-butylacrylamide/acrylamide), poly (butyl acrylate/n-butylacrylamide/acrylamide), poly(ethyl hexyl acrylate/n-butylacrylamide), poly(ethyl hexyl acrylate/n-butylacrylamide/acrylic acid), poly(ethyl hexyl acrylate/n-butylacrylamide/methyl methacrylate), poly(ethylhexyl acrylate/n-butyacrylamide/acrylic acid), poly(ethylhexyl acrylate/n-isopropylacrylamide/acrylic acid/methyl methacrylate), poly(n-isopropylacrylamide/dodecyl acrylate), poly(ethylhexyl acrylate/n-isopropylacrylamide/acrylic acid/methyl methacrylate/methacrylic acid), poly(stearyl acrylate, n-isopropylacrylamide/methyl methacrylate), poly(stearyl acrylate, n-isopropylacrylamide/methyl methacrylate), poly(stearyl acrylate/ethyl hexyl acrylate/n-isopropylacrylamide).

In one aspect, the copolymer comprises a residue of an alkyl methacrylamide and at least two C₁-C₁₈ alkyl acrylates. In another aspect, the copolymer comprises residues from n-isopropylmethacrylamide, dodecyl acrylate, and ethylhexyl acrylate. In another aspect, the copolymer comprises residues from n-isopropylmethacrylamide, dodecyl acrylate, and ethylhexyl acrylate, where the copolymer is crosslinked with a crosslinker (e.g. polyethyleneglycol diacrylate). In another aspect, the molar ratio of n-isopropylmethacrylamide to the sum of dodecyl acrylate, and ethylhexyl acrylate is 1:1 to 3:1, or 1:1, 1.5:1, 2:1, 2.5:1, or 3:1, where any value can be an lower and upper endpoint of a range (e.g., 2:1 to 2.5:1).

In certain aspects, additional monomers may be included in the copolymers to modify the film properties produced by the coating composition including, but not limited to, strength, appearance, adhesion, removal behavior and hardness. Selected examples include acrylic acid, 2-acrylamido-2-methyl-1-propanesulfonic acid, 4-styrenesulfonic acid tetrabutylammonium salt, methyl methacrylate, vinyl acetate, methacrylic acid, ethyl maleate, isopropyl maleate, butyl maleate, isobornyl acrylate, isobornyl methacrylate, styrene, n-isopropylmethacrylamide, and dimethyl aminoethyl methacrylate, or any combination thereof.

In one aspect, the copolymer includes at least one residue from the monomers in Group (a) and at least one residue from the monomers in Group (b):
a. acrylamide, methacrylamide, an alkyl acrylamide, a dialkyl acrylamide, an alkyl methacrylamide, a dialkyl methacrylamide, or any combination thereof; and
b. one or more alkyl acrylates.

In one aspect, the residue from Group (a) is from about 40 mole percent to about 90 mole percent of the copolymer. In another aspect, the residue from Group (a) is from about 40 mole percent, 45 mole percent, 50 mole percent, 55 mole percent, 60 mole percent, 65 mole percent, 70 mole percent, 75 mole percent, 80 mole percent, 85 mole percent, or 90 mole percent, where any value can be a lower and upper endpoint of a range (e.g., 50 mole percent to 75 mole percent).

In one aspect, the residue from Group (b) is greater than 0 mole percent to about 40 mole percent of the copolymer. In another aspect, the residue from Group (b) is greater than 0 mole % mole percent, 5 mole percent, 10 mole percent, 15 mole percent, 20 mole percent, 25 mole percent, 30 mole percent, 35 mole percent, or 40 mole percent, where any value can be a lower and upper endpoint of a range (e.g., 20 mole percent to 35 mole percent).

In one aspect, the copolymer is a thermoresponsive copolymer including a first monomer and a second monomer. In one aspect, the first monomer is N-isopropylacrylamide and the second monomer is a C1 to C10 alkyl acrylate, methacrylate, or mixtures thereof such as, for example, methyl acrylate, ethyl acrylate, ethylhexyl acrylate, propyl acrylate, butyl acrylate, stearyl acrylate, dodecyl acrylate, pentyl acrylate, methyl methacrylate, ethyl methacrylate, propyl methacrylate, butyl methacrylate, pentyl methacrylate, and the like.

In one aspect, the first monomer includes from about 99 mole percent to about 50 mole percent of the copolymer. In another aspect, the first monomer is from 99 mole percent to about 60 mole percent, about 99 mole percent to about 70 mole percent, or about 90 mole percent to about 40 mole percent of the copolymer.

In one aspect, the second monomer is from about 0.5 mole percent to about 40 mole percent of the copolymer. In another aspect, the second monomer is from about 5 mole percent to about 20 mole percent, about 10 mole percent to about 30 mole percent, or about 15 mole percent to about 35 mole percent of the copolymer.

In another aspect, a third monomer is incorporated in the copolymer, where the third monomer is different from the first and second monomer. In one aspect, the third monomer is selected from methyl methacrylate, acrylic acid, methacrylic acid, acrylamide, isobornyl methacrylate, hexyl acrylate, stearyl acrylate, dodecyl acrylate, or similar alkyl acrylates. In one aspect, the third monomer is from about 1 mole percent to about 40 mole percent of the copolymer. In another aspect, the third monomer is from about 1 to about 20 mole percent, about 10 mole percent to about 30 mole percent, or about 5 mole percent to about 15 mole percent.

In another aspect, a fourth monomer is incorporated in the copolymer, where the fourth monomer is different from the first, second, and third monomer. In one aspect, the fourth monomer is selected from methyl methacrylate, acrylic acid, methacrylic acid, acrylamide, isobornyl methacrylate, hexyl acrylate, stearyl acrylate, dodecyl acrylate, or similar alkyl acrylates. In one aspect, the fourth monomer is from about 1 mole percent to about 40 mole percent. In another aspect, the fourth monomer is from about 1 mole percent to about 20 mole percent, about 10 mole percent to about 30 mole percent, or about 5 mole percent to about 15 mole percent.

In certain aspects, one or more cross-linking monomers are included in the copolymers. These monomers have two or more vinyl functional groups that allow the linking of multiple polymer chains. Glycol diacrylates, other diacrylates, and bisacrylamides are examples of some linking monomers used. In some embodiments these monomers are comprised of polyethyleneglycol diacrylate, bisacrylamide and methylene bisacrylamide.

In certain aspects, the copolymers can be crosslinked prior to application, but only to an extent that they can still be easily applied and desirable appearance is not lost. The polymer structure is chosen to have a molecular chain length greater than its entanglement length.

In other aspects, the coating composition includes multiple copolymers comprised of a mixture of linear chains with a smaller proportion of cross-linked chains. In this case, the supporting cross-linked polymer may have up to 10 mol % covalent crosslinker in the polymer backbone.

In certain aspects, the copolymer has a weight average molecular weight of about 1,000 to about 1,000,000 Daltons. In some aspects, when two copolymers are used, the first copolymer and the second copolymer have a number average weight of about 1,000 to about 1,000,000 Daltons. In another aspect, the copolymer has a weight average molecular weight of about 10,000 to 300,000 Daltons.

In one aspect, the crosslinker is greater than about 0 mole percent to about 35 mole percent of the copolymer. In another aspect, the crosslinker is greater than about 0 mole percent, about 5 mole percent to about 20 mole percent, about 10 mole percent to about 30 mole percent, or about 15 mole percent to about 35 mole percent of the copolymer.

In one aspect, the copolymer is from about 5 weight percent to about 50 weight percent of the coating composition. In another aspect, the copolymer is from about 5 weight percent, about 10 weight percent, about 15 weight percent, about 20 weight percent, about 25 weight percent, about 30 weight percent, about 35 weight percent, about 40 weight percent, about 45 weight percent, or about 50 weight percent of the coating composition, where any value can be a lower and upper endpoint of a range (e.g., 25 weight percent to 45 weight percent).

The copolymers used in the coatings are prepared using several polymerization techniques including atom transfer radical polymerization, reversible addition fragmentation chain transfer, and free radical polymerization. Nonlimiting procedures making the copolymers are provided in the Examples.

### Solvents

The coating compositions described herein include a solvent that permits the formation of a film to form and provide the medium for the coating mixture. Not wishing to be bound by theory, the copolymer remains upon evaporation of the solvent to form covering layer along with any other non-volatile ingredients in the mixture. Some residual solvent may also remain trapped or absorbed in the polymer film.

In one aspect, the solvent is a polar organic solvent, water, or a combination thereof. In another aspect, the solvent is an alcohol such as, for example, ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, water, propylene glycol, glycerin, propylene carbonate, dimethyl glutarate, dimethyl adipate, methyl soyate, 2,2,4-trimethyl-1,3-pentanediol monoisobutyrate, denatured alcohol, diacetone alcohol, 1-methoxy, dimethoxy propanol, ethoxy propanol, or any combination thereof.

In certain aspects, a combination of two or more solvents can be used to modify the coating set time. A general set time can be estimated by comparing the evaporation rate of two solvents. For example, the combination of ethyl acetate and butyl acetate, 5.5 and 1 on a comparative scale to butyl acetate will allow for a relatively quick set, but still allow a decent working time during application.

In another aspect, the solvent includes a secondary solvent such as, for example, diacetone alcohol, 1-methoxy-2-propanol, propylene glycol, glycerin, propylene carbonate, dimethyl glutarate, dimethyl adipate, methyl soyate, and 2,2,4-trimethyl-1,3-pentanediol monoisobutyrate.

In one aspect, the solvent is from about 40 weight percent to about 90 weight percent of the coating composition. In another aspect, the solvent is from about 40 weight percent, about 45 weight percent, about 50 weight percent, about 55 weight percent, about 60 weight percent, about 65 weight percent, about 70 weight percent, about 75 weight percent, about 80 weight percent, about 85 weight percent, or about 90 weight percent of the coating composition, where any value can be a lower and upper endpoint of a range (e.g., 45 weight percent to 75 weight percent).

### Secondary Polymers

In certain aspects, the coating compositions described herein include one or more secondary polymers. Secondary polymers can provide additional features to the coating compositions such as, for example, strength, flexibility, gloss, hardness, pigment dispersion, or removal.

In one aspect, the secondary polymer is a polyester, an alkyd, a polyurethane, a tosylamide formaldehyde resin, an acrylic resin, vinyl acetate/butyl maleate/IBA copolymer, imidized isobutylene/maleic anhydride copolymer, polyvinyl methyl ether/maleic acid copolymer, a cellulose ester, a rosinate, or any combination thereof.

In one aspect, the secondary polymer is a cellulose ester, wherein the cellulose ester comprises cellulose acetate butyrate, nitrocellulose, cellulose acetate propionate, cellulose acetate phthalate, hydroxypropyl cellulose, hydroxyethyl cellulose, or any combination thereof. Each of these cellulose esters can be purchased or manufactured in a range of molecular weights and constituent ratios. In one aspect, the inclusion of the cellulose ester in the coating composition can provide additional gloss and strength to the resulting film.

In another aspect, other secondary polymers can improve the strength of a formula that are not of the vinyl and cellulose ester categories. These include polyesters, polyurethanes, silicones. Examples include poly(adipic acid/neopentyl glycol/trimellitic anhydride), poly (hexylene glycol/neopentyl glycol/adipic acid, saturated methylene diphenyldiisocyanate/dimethylolpropionic acid), amodimethicone, and dimethicone.

In one aspect, the secondary polymer is from about 0.5 weight percent to about 20 weight percent of the coating composition. In another aspect, the secondary polymer is from about 0.5 weight percent, about 2 weight percent, about 4 weight percent, about 6 weight percent, about 8 weight percent, about 10 weight percent, about 12 weight percent, about 14 weight percent, about 16 weight percent, about 18 weight percent, or about 20 weight percent of the coating composition, where any value can be a lower and upper endpoint of a range (e.g., 6 weight percent to 12 weight percent).

### Additives

The coating compositions described herein include one or more additives. The additives can modify physical properties, chemical properties, or health benefits of the coatings. In one aspect, the when the coating composition is for topical use, the additive can include a polymer, a rheology modifier, a suspension agent, a dispersant, a wetting agent, a low vapor pressure solvent, a plasticizer, pigments, effect powders, fragrance, an antimicrobial agent, an emollient, an emulsifier, a solubilizer, a humectant, a stabilizer, an exfoliant, a lipid or oil, a vitamin, a biological molecule, an essential oil, a fragrance, a preservative, a thickener, color additive, pH adjuster, a surfactant, or any combination thereof.

The amount of an additive in the total composition can vary. In one aspect, the weight % of one additive constitutes no more than 10% of the total coating formulation. In another aspect, the additive is from about 0.5 weight percent, about 1 weight percent, about 2 weight percent, about 3 weight percent, about 4 weight percent, about 5 weight percent, about 6 weight percent, about 7 weight percent, about 8 weight percent, about 9 weight percent, or about 10 weight percent of the coating composition, where any value can be a lower and upper endpoint of a range (e.g., 3 weight percent to 7 weight percent).

When the coating composition is used to provide a desired color or appearance, one or more pigments are incorporated into the coating composition. Examples of pigments include, but are not limited to, 3158TPLitholRubine BK, Carbon black N330, Degussa Printex, Titanium dioxide, D&C Brown No 1, D&C Black No 2, D&C Black No 3, 1104 Fast Yellow, 1283 Permanent Yellow HR, 1646 Medium Chrome yellow, FD&C Yellow No 5, D&C Yellow No 5 lake, D&C Yellow No 6 lake, D&C Yellow No 7 lake, D&C Yellow No 8, D&C Yellow No 10, D&C Yellow No 11, 3157 Vulcan Red LC, FD&C Red 3 lake, FD&C Red 4 lake, D&C Red No 4 aluminium lake, D&C Red No 6 aluminium lake, D&C Red No 6 barium lake, D&C Red No 7 lake, D&C Red No 8 lake, D&C Red No 9 lake, D&C Red No 17, D&C Red No 21, D&C Red No 22, D&C Red No 27 lake, D&C Red No 28, D&C red No 30 lake, D&C Red No 31, D&C Red No 33 lake, D&C red No 34 lake, D&C Red No 36 lake, FD&C Red No 40, 4421 Phthalocyanine Blue B pigment, 4431 Phthalocyanine Blue BS pigment, 4311 Pigment Sky Blue B, FD&C Blue 1 lake, D&C Blue No 4, 5319 Fast Green lake pigment, 5406 Phthalocyanine Green G pigment, 3327 Basic Rhodamine BG Lake pigment, FD&C Green No 3, D&C Green No 3 lake, D&C Green No 5, D&C Green No 6, D&C Green No 8, 6360 Fast Violet lake pigment, D&C Violet No 2, D&C Orange No 4 lake, D&C Orange No 5 aluminium lake, D&C Orange No 5 zirconium lake, D&C Orange No 10 aluminium lake, D&C Orange No 11, D&C Orange No 17 lake, Iron oxides of various shades, manganese violet, silver nitrate, ultramarines, zinc oxide, ferric ammonium ferrocyanide, ferric ferrocyanide, aluminum powder, bismuth oxychloride, copper powder, chromium hydroxide green, chromium oxide green, silver, Mica, or a combination thereof.

In other aspects, additional effect pigments are included such as micas, pearls, glitter, metal flakes glass, additional pigment or a combination thereof.

In one aspect, the pigment is from about 0.5 weight percent about 10% of the total weight coating composition. In another aspect, the pigment is from about 0.5 weight percent, about 1 weight percent, about 2 weight percent, about 3 weight percent, about 4 weight percent, about 5 weight percent, about 6 weight percent, about 7 weight percent, about 8 weight percent, about 9 weight percent, or about 10 weight percent of the coating composition, where any value can be a lower and upper endpoint of a range (e.g., 3 weight percent to 7 weight percent).

In one aspect, the pigment is in a dispersion. In one aspect, the pigment is first milled in a plasticizer (e.g., triethyl acetyl citrate) then dispersed into a pre-made suspension solution of silica and bentonite in an alcohol (e.g., ethanol).

In one aspect, suspension agents and rheology modifiers can be used to prevent settling of the pigment. In one aspect, a filler such as, for example, hectorite, stearalkonium hectorite, bentonite, bentonite, stearalkonium bentonite, silica, hydrophobic fumed silica, hydrophilic fumed silica, silica dimethyl silylate, organophilic phyllosilicate, kaolin clay, halloysite, montmorillonite, or any combination thereof can be used.

In one aspect, the filler includes glass microspheres. Not wishing to be bound by theory, the glass microspheres can reduce or prevent cracking of the coating composition once it has been applied. In another aspect, the filler includes barium sulfate. Not wishing to be bound by theory, barium sulfate can improve color homogeneity and opacity while reducing streaks in light colors.

In one aspect, the filler is from about 0.5 weight percent about 10% of the total weight coating composition. In another aspect, the filler is from about 0.5 weight percent, about 1 weight percent, about 2 weight percent, about 3 weight percent, about 4 weight percent, about 5 weight percent, about 6 weight percent, about 7 weight percent, about 8 weight percent, about 9 weight percent, or about 10 weight percent of the coating composition, where any value can be a lower and upper endpoint of a range (e.g., 3 weight percent to 7 weight percent).

In certain aspects, dispersants and wetting agents are used to improve the uniformity of coatings and prevent aggregation. Examples include, but not limited to, glycols, surfactants, ethoxylated alcohols, polymeric wetting agents like ethylene oxide-propylene oxide block copolymers, and silicones. In some embodiments the wetting agent in the coatings is styrene/acrylates copolymer, acrylic block copolymer, ethylene oxide/propylene oxide block copolymer, stearic acid, jojoba esters, glycerol, propylene glycol, ethylene glycol, lactonic sophorolipid, cocoamido propyl dimethyl amine, alpha-arbutin, cocamidopropylamine oxide, 2-octyldodecyl 2-ethylhexanoate, 1, 2, 3-trilinolenoylglycerol, cyclopentanecarboxylic acid sodium lauryl sulfate, octyl phenol ethoxylate, polysorbate 20, dimethicone, amodimethicone, or any combination thereof.

In certain aspects, low vapor pressure solvents are used to improve the coating durability, act as coalescing agents, provide shrink resistance, or alter the quality of application of the coating composition. Examples include, but not limited to, propylene carbonate, dimethyl glutarate, dimethyl adipate, methyl soyate, 2,2,4-trimethyl-1,3-pentanediol monoisobutyrate, dimethyl sulfoxide, or a combination thereof.

In certain aspects, plasticizers are incorporated to improve the flexibility or crack resistance of the coating. Examples include, but not limited to, acetyl tributyl citrate, tributyl citrate, triethyl citrate, dimethyl adipate, glycerin propylene glycol, ethylene glycol monoricinoleate, 2,2,4-trimethyl-1,3-pentanediol monoisobutyrate, bis(2-ethylhexyl) sebacate, diisopropyl sebacate, tridodecyl phosphate, dimethyl adipate, lavender essential oil, jojoba oil, argan oil, essential oils, plant oils, or a combination thereof. Some surfactants, resins, and other ingredients listed elsewhere in this disclosure with low vapor pressure are also suitable as plasticizers.

In one aspect, the additive is an anti-microbial agent including, but not limited to, tetrasodium EDTA, disodium EDTA, phenoxyethanol, diazolidinyl urea, iodopropynyl butylcarbamate, Lactobacillus & Cocos Nucifera (coconut) fruit extract, Leuconostoc ferment filtrate, benzyl alcohol, salicylic acid, glycerin, sorbic acid, gluconolactone, Sorbus aucuparia fruit ferment filtrate, capryl glycol, Sambucus nigra fruit extract, or any combination thereof.

In one aspect, the additive is an emollient including, but not limited to, shea butter, cocoa butter, mineral oil, lanolin, petrolatum, paraffin, beeswax, squalene, coconut, jojoba, sesame, avocado, argan, jojoba, almond, castor, and other plant oils(hydrogenated or unhydrogenated), cetyl alcohol, olive oil (e.g., oleic acid), triethylhexanoin, copernicia cerifera wax, lauryl laurate, capric triglyceride, cetearyl alcohol, alkyl benzoate, glyceryl stearate, coceth-20, glycerin, hydroxyetheyl cellulose, potassium sorbate, sodium phytate, citric acid, Lactobacillus ferment, behenyl alcohol, Nereocystis luetkeana ferment filtrate, hydrogenated polyisobutene, or any combination thereof.

In one aspect, the additive is an emulsifier including, but not limited to, cetearyl alcohol, glyceryl stearate, sodium stearoyl lactylate, PEG-100 stearate, behentrimonium methosulfate, polysorbate, sodium acrylate, isohexadecane, acacia gum, PEG, PEG-7 glyceryl monococoate, PEG-40 hydrogenated castor oil, PEG distearate, sorbitan laurate, polyglyceryl laurate, dilauryl citrate, caprylic triglyceride, cetearyth-n, sodium polyacrylate, ehthylhexyl cocoate, PPG-3 Benzyl ether myristate, or any combination thereof.

In one aspect, the additive is a solubilizer including, but not limited to, capryl glucoside, ethoxydiglycol, sodium cocoyl glutamate, glyceryl caprylate, polyglyceryl-6 oleate, sodium surfactin, D-glucopyranose, decyl octyl glycosides, polyglyceryl caprylate, polyglyceryl caprate, polyglyceryl cocoate, polyglyceryl ricinoleate, or any combination thereof.

In one aspect, the additive is a humectant including, but not limited to, propanediol, butylene glycol, ethoxy diglycol, ethylhexyl glycerin, pentylene glycol, propylene glycol, silica gel, PPG-20 methyl glucose ether, glycerin, malic acid, honey, honey powder, or any combination thereof.

In one aspect, the additive is a stabilizer including, but not limited to, xanthum gum, glucose oxidase, sclerotium gum, behenyl alcohol, cetyl alcohol, candellia wax, arrowroot powder, plant wax, sorbitan olivate, or any combination thereof.

In one aspect, the additive is an exfoliant including, but not limited to, salicylic acid, citric acid, glycolic acid, urea, plant powders, plant acids, glucosamine, plant extracts, enzymatic products of bacterial fermentation, or any combination thereof.

In one aspect, the additive is a binder selected from acrylic polymers, alkyds, polyurethanes, vinyl acetate, epoxy, polyvinyl chloride, latex, chitosan, casein, rubber acrylic resins, or combinations thereof.

In one aspect, the additive is a lipid or oil including, but not limited to, ceramides, blueberry seed oil, acacia seed oil, algae oil, almond oil, black currant extract, blackberry seed oil, borrage oil, jasmine oil, pomegranate seed oil, raspberry seed oil, other plants oils and extracts, cholesterol, phospholipids, linoleic acid, or combinations thereof.

In one aspect, the additive is a peptide, pre-peptide, or other biological molecule including, but not limited to, collagen pre-peptide, collagen, palmitoyl pentapeptide-4, palmitoyl oligopeptide, palmitoyl tetrapeptide-7, ferment peptide extracts, or plant extract derived peptides.

In one aspect, the additive is a surfactant. In another aspect, the surfactant is a nonionic surfactant. Examples of nonionic surfactants include the condensation products of a higher aliphatic alcohol, such as a fatty alcohol, containing about 8 to about 20 carbon atoms, in a straight or branched chain configuration, condensed with about 3 to about 100 moles, preferably about 5 to about 40 moles, most preferably about 5 to about 20 moles of ethylene oxide. Examples of such nonionic ethoxylated fatty alcohol surfactants are the Tergitol™ 15-S series from Union Carbide and Brij™ surfactants from ICI. Tergitol™ 15-S Surfactants include C11-C15 secondary alcohol polyethyleneglycol ethers. Brij™97 surfactant is polyoxyethylene(10) oleyl ether; Brij™58 surfactant is polyoxyethylene(20) cetyl ether; and Brij™ 76 surfactant is polyoxyethylene(10) stearyl ether.

Another useful class of nonionic surfactants include the polyethylene oxide condensates of one mole of alkyl phenol containing from about 6 to 12 carbon atoms in a straight or branched chain configuration, with ethylene oxide. Examples of nonreactive nonionic surfactants are the Igepal™ CO and CA series from Rhone-Poulenc. Igepal™CO surfactants include nonylphenoxy poly(ethyleneoxy)ethanols. Igepal™ CA surfactants include octylphenoxy poly(ethyleneoxy)ethanols.

Another useful class of hydrocarbon nonionic surfactants include block copolymers of ethylene oxide and propylene oxide or butylene oxide. Examples of such nonionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers.

In other aspects, the nonionic surfactants include sorbitan fatty acid esters, polyoxyethylene sorbitan fatty acid esters and polyoxyethylene stearates. Examples of such fatty acid ester nonionic surfactants are the Span™, Tween™, and Myj™ surfactants from ICI. Span™ surfactants include C12-C18 sorbitan monoesters. Tween™ surfactants include poly(ethylene oxide) C12-C18 sorbitan monoesters. Myj™ surfactants include poly(ethylene oxide) stearates.

In one aspect, the nonionic surfactant can include polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ether, polyoxyethylene cetyl ether, polyoxyethylene stearyl ether, polyoxyethylene oleyl ether, polyoxyethylene octylphenyl ether, polyoxyethylene nonylphenyl ether, polyethylene glycol laurate, polyethylene glycol stearate, polyethylene glycol distearate, polyethylene glycol oleate, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurate, polyoxyethylene sorbitan stearate, polyoxyethylene sorbitan oleate, polyoxyethylene laurylamine, polyoxyethylene laurylamide, laurylamine acetate, hard beef tallow propylenediamine dioleate, ethoxylated tetramethyldecynediol, fluoroaliphatic polymeric ester, polyether-polysiloxane copolymer, and the like.

In one aspect, a pH adjuster is included in the coating compositions described herein. Examples include, but are not limited to, citric acid, sodium bicarbonate, alkyl siliconates, magnesium hydroxide, triethanolamine, ammonia, aminomethyl-propanol, sugar-amines, protonated sugar amines, or combinations thereof.

In one aspect a color protectant is included in the coating compositions described herein. Examples include, but are not limited to, hydroxyphenyl benzotriazoles, butanedioic acid dimethylester polymer with 4-hydroxy-2,2,6,6-tetramethyl-1-piperidine ethanol, other oligomeric UV stabilizers, bisoctrizole, bemotrizinol, dimethyl (p-methoxybenzylidene)malonate, dioxybenzone, hindered amines, high molecular weight oligomeric hindered amines, assorted antioxidants, hydroxyacetophenone, butylated hydroxytoluene, butylated hydroxyanisole, vitamin E, other vitamins, plant extracts, green tea extract, rosemary extract, resveratrol, UV absorbers, benzophenone, ethylhexyl methoxycinnamate, ethylhexyl salicylate, avobenzone, homosalate, octocrylene, and combinations thereof.

In another aspect, the coating compositions described herein as well as other coatings (e.g., base coating, protective coating) discussed in greater detail below can include additional ingredients to improve the scent of the coatings. These include synthetic and natural fragrances. In some embodiments they include essential oils, agrumen, amber, ambergris, ambrette, amyris, benzoin, bergamot, calone, cashmeran, castoreum, citron, civet, clary sage, coumarin, frangipani, frankincense, galbanum, guaiac wood, hedione, heliotrope, iso e super, jasmine, jasmine sambac, labdanum, mimoa, monoi, muguet, musk, myrrh, narcissus, neroli, oakmoss, opopanax, orris, oud, ozone, patchouli, rose, rose de mai, sandalwood, tonka bean, tuberose, anilla, vetiver, ylang ylang, cedarwood, cardamom, lavender, tea tree, lemongrass, eucalyptus, hydrosol, plant water, or combination thereof.

In certain aspects, any of the coatings described herein can include synthetic fragrances, ethylvanillin, heliotropine, ionones, aldehydes, calone, ambrox, ethyl maltol, myristic acid, 2-furaldehyde, delta decalactone, delta dodecalactone, 2 methyl 2 pentenoic acid, 2,4 decadienal, 5 methyl 2 phenyl 2 hexenal, acetaldehyde, acetyl propionyl, anisyl, acpric acid, capoic acid, caprylic acid, cyclotene, methyl 3 nonenoate, ethyl 3 hydroxybutyrate, ethyl maltol, lactic acid, lauric acid, pulegone mercaptan,

4-(p-hydroxyphenyl)-2-butanone, 4-hydroxy-2,5-dimethyl-3(2H)-furanone, triethyl citrate, furanone, acetoin, 6-methyl coumarin, 4,5-dimethyl-3-hydroxy-2,5-dihydrofuran-2-one, 2,5 dimethyl 4-methoxy-3(2h)-furanone, 2-methoxy-4-methylphenol, 5-ethyl-3-hydroxy-4-methyl-2(5h)furanone, caproic acid methyl ester, oleic acid ethyl ester, methyl salicylate, 4-hydroxy pentanoic acid y lactone, 5-ethyl-3-hydroxy-4-methyl-2(5h)furanone, 1,5,5,9-tetramethyl-13-oxatricyclo(8.3.0.0)tridecane, isobutyric acid maltol ester, diethyl succinate, phenyl ethyl isothiocyanatem, 4 hydroxy-5-methyl-3-(2h) furanone, 2-ethyl-5-methyl-4-hydroxy-3(2H)-furanone, dihydro coumarin, ethyl 3-hydroxy caproate, furanone acetate, glyceryl triacetate, 1-octen-3-ol, 3-hydroxy-2-butanone, popcorn pyrimidine, 4-hydroxy-2,5-dimethyl-3(2H)-furanone, 5-methyl-2-hepten-4-one, 4-hydroxy-3methyl octanoic acid lactone, 2-buten-1-one, 1-(2,6,6 trimethyl-1,3cyclohexadien-1-yl, 3-cyclohexene-1carboxaldehyde, 2,4-dimethyl, butyl butyryl lactate, 1,2-di-[(1-ethoxy) ethoxy] propane, 4-(2,6,6-trimethyl cyclo-1-enyl)but-2-en-4-one, butanedioic acid, terpineol, 3-methyl-5-cyclopentadecen-1-one, 5-methyl-2-propan-2-ylhex-2-enal, related aromatic compounds, or any combination thereof.

In certain aspect, oils, vitamins, peptides, proteins, and other ingredients beneficial for health are incorporated into the coatings. In some embodiments these include alpha hydroxy acids, hyaluronic acid, retinol, salicylic acid, ceramides, sulfur, vitamin A, vitamin e, vitamin c, vitamin b, vitamin k, vitamin D, alpha-linolenic acid (ALA), eicosapentaenoic acid (EPA), and docosahexaenoic acid (DHA), calcium carbonate, calcium citrate, keratin, collagen, olive oil, rapeseed oil, jojoba oil, argan oil, essential oils, plant extracts, zinc gluconate, iron (II) sulfate, magnesium oxide, Phytosphingosine, tocopherol acetate, ashwagandha, curcumin, amino acids, peptides, piperine, chavicine, keratin, horsetail extract, amla extract, chamomile extract, sea buckthorn oil, sea buckthorn extract, centella asiatic extract, pin bark extract, plum extract, coffeeberry extract, and combinations thereof.

### Preparation of the Coating Compositions

The coating compositions and other coatings (e.g., base and protective coatings) are produced by admixing all ingredients into the solvent. In one aspect, all components are mixed for a sufficient time such that all components are dissolved or substantially dissolved in the solvent.

### Applications of Coating Compositions

The coating compositions described herein can be used as protective or decorative layers for a range of surfaces and substrates. They may also be used for alternative functions such as reduction of friction or delivery of therapeutic agents. Many embodiments of these coatings offer advantages over currently marketed coatings by removing harmful solvents, reducing odor, or facilitating easier removal. They also allow easier incorporation of certain therapeutic or health-oriented ingredients. In other aspects, the coating compositions can be re-applied to a previously coated substrate where the previous coating has chipped or faded.

The coatings can be applied to any surface using a number of techniques and devices. Since the coatings are a liquid or flowable substance prior to application to the skin surface, the coatings can be applied using a dropper, pressurized dispenser, aerosol dispenser, brush, spray coating or any other suitable devices and methods for delivering liquids to a surface.

In one aspect, two or more layers of the coating compositions described herein can be layered to achieve a desired effect such as sufficient, hardness, gloss, or environmental resistance.

The coating compositions described herein can be applied to a number of different substrates. In one aspect, the coating composition can be applied to a non-living substrate or article such as, for example, a vehicle or building. In another aspect, the substrate can be on a living subject such as, for example, a fingernail or toenail.

In one aspect, after application to a substrate, the coating composition can by dry to touch in under 10 minutes and achieve a set within 60% of final hardness within 3 hours.

### Base Coat

In certain aspects, a surface or substrate is coated with a base coat composition before applying the coating composition. In another aspect, a surface or substrate is coated with two or more base coat compositions before applying the coating composition. The base coat provides consistent performance of the coating composition across different types of surfaces. The base coat can also facilitate greater uniformity, gloss, or durability with later coats that are applied.

In one aspect, the base coat composition includes a solvent and a vinyl polymer, a polymer resin, or a combination thereof. In one aspect, the vinyl polymer can be any one of the polymers described above present in the coating composition. In one aspect, the polymer resin can be any of the secondary polymers described herein. In one aspect, the polymer resin is a polyester resin. In another aspect the base coat composition includes a vinyl polymer and a polyester resin.

In one aspect, the vinyl polymer is from about 5 weight percent to about 50 weight percent of the base coat. In another aspect, the vinyl polymer is from about 5 weight percent, about 10 weight percent, about 15 weight percent, about 20 weight percent, about 25 weight percent, about 30 weight percent, about 35 weight percent, about 40 weight percent, about 45 weight percent, or about 50 weight percent of the base coat, where any value can be a lower and upper endpoint of a range (e.g., 15 weight percent to 30 weight percent).

In another aspect, the polymer resin is from about 0.5 weight percent to about 10 weight percent of the base coat. In another aspect, the polymer is from about 0.5 weight percent, about 1 weight percent, about 2 weight percent, about 3 weight percent, about 4 weight percent, about 5 weight percent, about 6 weight percent, about 7 weight percent, about 8 weight percent, about 9 weight percent, or about 10 weight percent of the base coat, where any value can be a lower and upper endpoint of a range (e.g., 2 weight percent to 6 weight percent).

The base coat compositions described herein include a solvent that permits the formation of a film to form and provide the medium for the coating mixture. In one aspect, the solvent is a polar organic solvent, water, or a combination thereof. In another aspect, the solvent is an alcohol such as, for example, ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, or any combination thereof.

In one aspect, the base coat solvent is from about 40 weight percent to about 90 weight percent of the base coating composition. In another aspect, the solvent is from about 40 weight percent, about 45 weight percent, about 50 weight percent, about 55 weight percent, about 60 weight percent, about 65 weight percent, about 70 weight percent, about 75 weight percent, about 80 weight percent, about 85 weight percent, or about 90 weight percent of the base coating composition, where any value can be a lower and upper endpoint of a range (e.g., 45 weight percent to 75 weight percent).

The base coat composition can include one or more of the additives described above for the coating compositions. In one aspect, the base coat composition includes a therapeutic agent selected from the group consisting of a vitamin, protein, polysaccharide, salt, peptide, plant extract, oil, hydrosol, or any combination thereof. In another aspect, the base coat composition includes a humectant, an exfoliant, a dispersant, a suspension agent, a preservative, a thickener, an acid, a base, an enzyme, a filler, a color additive, a pH adjuster, a surfactant, a salt, a reactive species, or any combination thereof.

### Protective Coating

In another aspect, to improve the lifetime and appearance of the applied coating composition, a protective coating can be applied to the coating produced by the coating composition. In another aspect, two or more protective coatings can be applied to the coating produced by the coating composition. In one aspect, after application to a substrate with the coating produced from the coating composition, the protective coating can by dry to touch in under 10 minutes and achieve a set within 60% of final hardness within 3 hours.

In one aspect, the protective coating composition comprises a protective coating solvent and a cellulose ester or alkoxylated cellulose. In one aspect, the cellulose ester comprises cellulose acetate butyrate, ethyl cellulose, cellulose acetate propionate, cellulose acetate phthalate, or nitrocellulose.

In one aspect, the cellulose ester is from about 5 weight percent to about 30 weight percent of the protective coating composition. In another aspect, the vinyl polymer is from about 5 weight percent, about 10 weight percent, about 15 weight percent, about 20 weight percent, about 25 weight percent, or about 30 weight percent of the protective coating composition, where any value can be a lower and upper endpoint of a range (e.g., 10 weight percent to 25 weight percent).

In another aspect, the protective coating composition can further include a secondary polymer as described herein. In one aspect, the secondary polymer selected from adipic acid/neopentyl glycol/trimellitic anhydride copolymer, hexanedioic acid, a polycondensate of adipic acid/(esterified reaction product of glycerin and coconut oil fatty acid)/2,2′-(27-ethylamino)diethanol, tridecyl trimellitate, an acrylic polymer, vinyl acetate/butyl maleate/IBA, imidized isobutylene/maleic anhydride copolymer, polyvinyl methyl ether/maleic acid copolymer, or any combination thereof.

The protective coating compositions described herein include a solvent that permits the formation of a film to form and provide the medium for the coating mixture. In one aspect, the solvent is a polar organic solvent, water, or a combination thereof. In another aspect, the solvent is an alcohol such as, for example, ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, water, propylene glycol, glycerin, propylene carbonate, dimethyl glutarate, dimethyl adipate, methyl soyate, 2,2,4-trimethyl-1,3-pentanediol monoisobutyrate, denatured alcohol, diacetone alcohol, 1-methoxy, dimethoxy propanol, ethoxy propanol, or any combination thereof.

In one aspect, the protective coating solvent is from about 40 weight percent to about 90 weight percent of the protective coating composition. In another aspect, the solvent is from about 40 weight percent, about 45 weight percent, about 50 weight percent, about 55 weight percent, about 60 weight percent, about 65 weight percent, about 70 weight percent, about 75 weight percent, about 80 weight percent, about 85 weight percent, or about 90 weight percent of the protective coating composition, where any value can be a lower and upper endpoint of a range (e.g., 45 weight percent to 75 weight percent).

The protective coating composition can include one or more of the additives described above for the coating compositions. In one aspect, the protective coating composition includes a volatile solvent, a low vapor pressure solvent, acrylic polymer, a plasticizer, a humectant, a dispersant, a suspension agent, a lipid, an oil, a vitamin, a mineral, a biological molecule, an essential oil, a plant extract, a plant hydrosol, a fragrance, a preservative, a thickener, a color additive, a pH adjuster, a surfactant, or any combination thereof.

### Removal Solution

Due to the components present in the coating compositions described herein, the resulting coating can be readily removed from the substrate. In one aspect, a removal solution composed of an organic solvent, water, or a combination thereof can be used to remove the coating. The removal solution permits the removal of the coatings described herein from any biological surface in a way that reduces potential harm or provides therapeutic or cosmetic benefit.

In one aspect, the removal solution is odorless. This feature is very different from 100% acetone typically used to remove coatings such nail polish and the like. Not wishing to be bound by theory, the copolymers described herein are soluble in a variety of alcohols, which permits their removal using odorless solvents.

In one aspect, the organic solvent in the removal solution comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, water, propylene glycol, glycerin, propylene carbonate, dimethyl glutarate, dimethyl adipate, methyl soyate, 2,2,4-trimethyl-1,3-pentanediol monoisobutyrate, denatured alcohol, diacetone alcohol, 1-methoxy, dimethoxy propanol, ethoxy propanol, or any combination thereof.

In one aspect, the removal solution does not include additional additives such as, for example, include water, propylene carbonate, dimethyl adipate, methyl soyate, and 2,2,4-trimethyl-1,3-pentanediol monoisobutyrate, polyols, a vitamin, a hydrosol, a plant extract, a biological molecule, a protein, an essential oil, a fragrance, a preservative, or any combination thereof.

Due to the polar nature of the removal facilitates incorporation of previously listed beneficial ingredients. In one aspect, the removal solution includes propylene carbonate, dimethyl glutarate, dimethyl adipate, methyl soyate, 2,2,4-trimethyl-1,3-pentanediol monoisobutyrate, dimethyl sulfoxide, or any combination thereof. In another aspect, the removal solution includes a therapeutic agent selected from the group consisting of a vitamin, protein, polysaccharide, salt, peptide, plant extract, oil, hydrosol, or any combination thereof. In another aspect, the removal solution includes a humectant, an exfoliant, a dispersant, a suspension agent, a preservative, a thickener, an acid, a base, an enzyme, a filler, a color additive, a pH adjuster, a surfactant, a salt, a reactive species, or any combination thereof.

### Kits

The coating compositions and other compositions described herein (e.g., base coating composition, protective coating composition, removal solution) can be manufactured and sold as separate components.

In another aspect, the coating compositions can be part of a kit. In one aspect, the kit includes one or more coating compositions as described herein and an applicator. In one aspect, the applicator can include one or more brushes, stencils, stamps, or stickers. In another aspect, the kit can include a device for abrading the surface of the substrate prior to applying the coating composition.

In another aspect, the kit can include the base coating composition, protective coating composition, removal solution, or any combination thereof.

### ASPECTS

Aspect 1. A coating composition comprising a solvent and a copolymer comprising at least one residue from the monomers in Group (a) and at least one residue from the monomers in Group (b):
   a. acrylamide, methacrylamide, an alkyl acrylamide, a dialkyl acrylamide, an alkyl methacrylamide, a dialkyl methacrylamide, or any combination thereof; and
   b. one or more alkyl acrylates.
Aspect 2. The coating composition of Aspect 1, wherein the Group (a) monomer is selected from the group consisting of n-methyl acrylamide, n-ethylacrylamide, n-isopropylacrylamide, n-butylacrylamide, n-tertbutylacrylamide, n-methylmethacrylamide, n-ethyl methacrylamide, n-isopropyl methacrylamide, n-butyl methacrylamide, n-tertbutyl methacrylamide, and any combination thereof.
Aspect 3. The coating composition of Aspect 1, wherein the alkyl acrylate is a C₆-C₁₈ alkyl acrylate.
Aspect 4. The coating composition of Aspect 1, wherein the alkyl acrylate is selected from the group consisting of methyl acrylate, ethyl acrylate, propyl acrylate, butyl acrylate, pentyl acrylate, hexyl acrylate, 2-ethylhexyl acrylate, styrene acrylate, heptyl acrylate, octyl acrylate, nonyl acrylate, decyl acrylate, undecyl acrylate, dodecyl acrylate, hexadecyl acrylate, stearyl acrylate, lauryl acrylate, t-butyl acrylate, isobutyl acrylate, isobornyl acrylate and any combination thereof.
Aspect 5. The coating composition of any one of Aspects 1-4, wherein the copolymer further comprises a residue from acrylic acid, 2-acrylamido-2-methyl-1-propanesulfonic acid, 4-styrenesulfonic acid tetrabutylammonium salt, methyl methacrylate, vinyl acetate, methacrylic acid, ethyl maleate, isopropyl maleate, butyl maleate, isobornyl acrylate, isobornyl methacrylate, styrene, n-isopropylmethacrylamide, and dimethyl aminoethyl methacrylate, or any combination thereof.
Aspect 6. The coating composition of Aspect 1, wherein the copolymer comprises residues from n-isopropylacrylamide, n-isopropyl methacrylamide, and an alkyl acrylate.
Aspect 7. The coating composition of Aspect 1, wherein the copolymer comprises residues from n-isopropylacrylamide, butyl acrylate, and methyl methacrylate.
Aspect 8. The coating composition of Aspect 1, wherein the copolymer comprises residues from n-isopropylmethacrylamide, dodecyl acrylate, methyl methacrylate, and acrylic acid.
Aspect 9. The coating composition of Aspect 1, wherein the copolymer comprises residues from n-isopropylmethacrylate, methyl methacrylate and ethylhexyl acrylate.
Aspect 10. The coating composition of Aspect 1, wherein the copolymer comprises residues from n-isopropylmethacrylate, ethylhexyl acrylate, and acrylic acid.
Aspect 11. The coating composition of any one of Aspects 1-10, wherein the copolymer is covalently crosslinked.
Aspect 12. The coating composition of Aspect 11, wherein the covalent crosslinker is a bis-acrylamide compound or a bis-acrylate compound.
Aspect 13. The coating composition of Aspect 11, wherein the covalent crosslinker is selected from the group consisting of methylenebisacrylamide, polyethyleneglycol diacrylate, ethylene glycol dimethacrylate, N-(1-hydroxy-2,2-dimethoxyethyl)acrylamide, divinylbenzene, phenylenediacrylamide, diurethane dimethacrylate, and any combination thereof.
Aspect 14. The coating composition of any one of Aspects 1-13, wherein the residue from Group (a) is from about 40 mole percent to about 90 mole percent of the polymer.
Aspect 15. The coating composition of any one of Aspects 1-14, wherein the residue from Group (b) is greater than 0 mole percent to about 40 mole percent of the polymer.
Aspect 16. The coating composition of any one of Aspects 1-15, wherein the copolymer further comprises a residue from acrylic acid, methyl methacrylate, methacrylic acid, and dimethyl aminoethyl methacrylate, or any combination thereof in the amount greater than 0 mole % to about 35 mole percent.
Aspect 17. The coating composition of any one of Aspects 1-16, wherein the copolymer further comprises a covalent crosslinker in the amount greater than 0 mole % to about 35 mole percent.
Aspect 18. The coating composition of any one of Aspects 1-17, wherein the copolymer has an average molecular weight from about 1,000 Da to about 1,000,000 Da.
Aspect 19. The coating composition of any one of Aspects 1-18, wherein the copolymer is from about 5 weight percent to about 50 weight percent of the coating composition.
Aspect 20. The coating composition of any one of Aspects 1-19, wherein the solvent comprises a polar organic solvent, water, or a combination thereof.
Aspect 21. The coating composition of any one of Aspects 1-19, wherein the solvent comprises an alcohol.
Aspect 22. The coating composition of any one of Aspects 1-19, wherein the solvent comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, or any combination thereof.
Aspect 23. The coating composition of any one of Aspects 19-22, wherein the solvent further comprises diacetone alcohol, 1-methoxy-2-propanol, propylene glycol, glycerin, propylene carbonate, dimethyl glutarate, dimethyl adipate, methyl soyate, and 2,2,4-trimethyl-1,3-pentanediol monoisobutyrate.
Aspect 24. The coating composition of any one of Aspects 1-23, wherein the solvent is from about 40 weight percent to about 90 weight percent of the coating composition.
Aspect 25. The coating composition of any one of Aspects 1-24, wherein the coating composition further comprises a secondary polymer comprising a polyester, an alkyd, a polyurethane, a tosylamide formaldehyde resin, an acrylic resin, vinyl acetate/butyl maleate/IBA copolymer, imidized isobutylene/maleic anhydride copolymer, polyvinyl methyl ether/maleic acid copolymer, a cellulose ester, a rosinate, or any combination thereof.
Aspect 26. The coating composition of Aspect 25, wherein the coating composition further comprises a secondary polymer comprising an ester or polyester, wherein the ester or polyester comprises adipic acid/neopentyl glycol/trimellitic anhydride copolymer, hexanedioic acid, a polycondensate of adipic acid/(esterified reaction product of glycerin and coconut oil fatty acid)/2,2′-(32ethylamino)diethanol, tridecyl trimellitate, or any combination thereof.
Aspect 27. The coating composition of Aspect 25, wherein the coating composition further comprises a secondary polymer comprising a cellulose ester, wherein the cellulose ester comprises cellulose acetate butyrate, nitrocellulose, cellulose acetate propionate, cellulose acetate phthalate, hydroxypropyl cellulose, hydroxyethyl cellulose or any combination thereof.
Aspect 28. The coating composition of any one of Aspects 25-27, wherein the secondary polymer is from about 0.5 weight percent to about 20 weight percent of the coating composition.
Aspect 29. The coating composition of any one of Aspects 1-28, wherein the coating composition further comprises a filler.
Aspect 30. The coating composition of Aspect 29, the filler comprises hectorite, stearalkonium hectorite, bentonite, bentonite, stearalkonium bentonite, silica, hydrophobic fumed silica, hydrophilic fumed silica, silica dimethyl silylate, organophilic phyllosilicate, kaolin clay, halloysite, montmorillonite, or any combination thereof.
Aspect 31. The coating composition of Aspect 29, wherein the filler comprises fumed silica and stearalkonium bentonite.
Aspect 32. The coating composition of any one of Aspects 29-31, wherein the filler is from about 0.5 weight percent to about 10 weight percent of the coating composition.
Aspect 33. The coating composition of any one of Aspects 1-32, wherein the coating composition further comprises a pigment.
Aspect 34. The coating composition of Aspect 33, wherein the pigment is from about 0.5 weight percent to about 10 weight percent of the coating composition.
Aspect 35. The coating composition of any one of Aspects 1-34, wherein the coating composition further comprises a plasticizer, a humectant, a dispersant, a suspension agent, a lipid, an oil, a vitamin, a biological molecule, an essential oil, a plant extract, a plant hydrosol, a fragrance, a preservative, a thickener, one or more color additives, a pH adjuster, a surfactant, or any combination thereof.
Aspect 36. A kit comprising the coating composition of any one of Aspects 1-35 and an applicator.
Aspect 37. The kit of Aspect 36, wherein the kit further comprises a protective coating composition.
Aspect 38. The kit of Aspect 37, wherein the protective coating composition comprises a protective coating solvent and a cellulose ester.
Aspect 39. The kit of Aspect 37, wherein the protective coating solvent comprises a polar organic solvent, water, or a combination thereof.
Aspect 40. The kit of Aspect 37, wherein the protective coating solvent comprises an alcohol.
Aspect 41. The kit of Aspect 37, wherein the protective coating solvent comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, or any combination thereof.
Aspect 42. The kit of Aspect 37, wherein the protective coating solvent comprises ethyl alcohol, diacetone alcohol, 1-methoxy-2-propanol, or a combination thereof.
Aspect 43. The kit of any one of Aspects 37-42, wherein the protective coating solvent is from about 40 weight percent to about 90 weight percent of the protective coating composition.
Aspect 44. The kit of any one of Aspects 37-42, wherein the cellulose ester comprises cellulose acetate butyrate, cellulose acetate propionate, cellulose acetate phthalate, or nitrocellulose.
Aspect 45. The kit of any one of Aspects 37-44, wherein the cellulose ester is from about 5 weight percent to about 30 weight percent of the protective coating composition.
Aspect 46. The kit of any one of Aspects 37-45, wherein the protective coating composition further comprises a secondary polymer selected from adipic acid/neopentyl glycol/trimellitic anhydride copolymer, hexanedioic acid, a polycondensate of adipic acid/(esterified reaction product of glycerin and coconut oil fatty acid)/2,2′-(34-ethylamino)diethanol, tridecyl trimellitate, an acrylic polymer, vinyl acetate/butyl maleate/IBA, imidized isobutylene/maleic anhydride copolymer, polyvinyl methyl ether/maleic acid copolymer, or any combination thereof.
Aspect 47. The kit of Aspect 46, wherein the polymer is from about 0.5 weight percent to about 25 weight percent of the protective coating composition.
Aspect 48. The kit of any one of Aspects 37-47, wherein the protective coating composition further comprises a volatile solvent, a low vapor pressure solvent, acrylic polymer, a plasticizer, a humectant, a dispersant, a suspension agent, a lipid, an oil, a vitamin, a mineral, a biological molecule, an essential oil, a plant extract, a plant hydrosol, a fragrance, a preservative, a thickener, a color additive, a pH adjuster, a surfactant, or any combination thereof.
Aspect 49. The kit of any one of Aspects 36-48, wherein the kit further comprises a removal composition comprising an organic solvent, water, or a combination thereof.
Aspect 50. The kit of Aspect 49, wherein the organic solvent comprises an alcohol.
Aspect 51. The kit of Aspect 49, wherein the organic solvent comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, or any combination thereof.
Aspect 52. The kit of Aspect 49, wherein the organic solvent comprises a mixture of ethyl alcohol and water.
Aspect 53. The kit of any one of Aspects 49-52, wherein the removal solution further comprises propylene carbonate, dimethyl glutarate, dimethyl adipate, methyl soyate, 2,2,4-trimethyl-1,3-pentanediol monoisobutyrate, dimethyl sulfoxide, or any combination thereof.
Aspect 54. The kit of any one of Aspects 49-53, wherein the removal solution further comprises a therapeutic agent selected from the group consisting of a vitamin, protein, polysaccharide, salt, peptide, plant extract, oil, hydrosol, or any combination thereof.
Aspect 55. The kit of any one of Aspects 49-54, wherein the removal solution further comprises a humectant, an exfoliant, a dispersant, a suspension agent, a preservative, a thickener, an acid, a base, an enzyme, a filler, a color additive, a pH adjuster, a surfactant, a salt, a reactive species, or any combination thereof.
Aspect 56. The kit of any one of Aspects 36-55, wherein the kit further comprises a base coat composition.
Aspect 57. The kit of Aspect 56, wherein the base coat composition comprises a solvent and a vinyl polymer, a polymer resin, or any combination thereof.
Aspect 58. The kit of Aspect 57, wherein the base coat solvent comprises a polar organic solvent, water, or a combination thereof.
Aspect 59. The kit of Aspect 57, wherein the base coat solvent comprises an alcohol.
Aspect 60. The kit of Aspect 57, wherein the base coat solvent comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, or any combination thereof.
Aspect 61. The kit of Aspect 57, wherein the base coat solvent comprises ethyl alcohol, diacetone alcohol, 1-methoxy-2-propanol, or a combination thereof.
Aspect 62. The kit of any one of Aspects 57-61, wherein the base coat solvent is from about 40 weight percent to about 90 weight percent of the base coat.
Aspect 63. The kit of any one of Aspects 57-61, wherein the base coat composition further comprises a therapeutic agent selected from the group consisting of a vitamin, protein, polysaccharide, salt, peptide, plant extract, oil, hydrosol, or any combination thereof.
Aspect 64. The kit of any one of Aspects 57-61, wherein the base coat composition further comprises a humectant, an exfoliant, a dispersant, a suspension agent, a preservative, a thickener, an acid, a base, an enzyme, a filler, a color additive, a pH adjuster, a surfactant, a salt, a reactive species, or any combination thereof.
Aspect 65. The kit of any one of Aspects 57-64, wherein the polymer resin is from about 0.5 weight percent to about 10 weight percent of the base coat composition.
Aspect 66. The kit of any one of Aspects 57-65, wherein the vinyl polymer is from about 5 weight percent to about 50 weight percent of the base coat composition.
Aspect 67. The kit of any one of Aspects 36-66, wherein the applicator comprises one or more brushes, stencils, stamps, or stickers.
Aspect 68. A method for coating a substrate, the method comprising applying the coating composition of any one of Aspects 1-35 to at least one surface of the substrate to produce a coated substrate.
Aspect 69. The method of Aspect 68, wherein the substrate is a vehicle or a building.
Aspect 70. The method of Aspect 68, wherein the substrate is a body part of a subject.
Aspect 71. The method of Aspect 68, wherein the substrate is a fingernail or toenail of a subject.
Aspect 72. The method of any one of Aspects 68-71, wherein the coating composition is applied by brushing, dipping, or spray coating the coating composition on the substrate.
Aspect 73. The method of any one of Aspects 68-72, wherein the method further comprises applying a protective coating composition to the coated substrate.
Aspect 74. The method of any one of Aspects 68-73, wherein the method further comprises applying a base coating composition to the substrate prior to applying the coating composition.
Aspect 75. A coated substrate produced by the method of any one of Aspects 68-74.
Aspect 76. A copolymer comprising at least one residue from the monomers in Group (a), at least one residue from the monomers in Group (b), and at least one residue from the monomers in Group (c):
   a. acrylamide, methacrylamide, an alkyl acrylamide, a dialkyl acrylamide, an alkyl methacrylamide, a dialkyl methacrylamide, or any combination thereof; and
   b. one or more alkyl acrylates, and
   c. acrylic acid, methyl methacrylate, methacrylic acid, and dimethyl aminoethyl methacrylate, or any combination thereof.
Aspect 77. The copolymer of Aspect 76, wherein the group (a) residue is an alkyl acrylamide.
Aspect 78. The copolymer of Aspect 76, wherein the group (a) residue is n-isopropyl acrylamide.
Aspect 79. The copolymer of any one of Aspects 76-78, wherein the group (b) residue is C₂ to C₁₀ alkyl acrylate.
Aspect 80. The copolymer of any one of Aspects 76-78, wherein the group (b) residue is ethylhexyl acrylate.
Aspect 81. The copolymer of any one of Aspects 76-80, wherein the group (c) residue is acrylic acid.
Aspect 82. The copolymer of any one of Aspects 76-80, wherein the group (c) residue is methyl methacrylate.
Aspect 83. The copolymer of any one of Aspects 76-82, wherein the polymer is covalently crosslinked.
Aspect 84. The copolymer of Aspect 83, wherein the polymer is covalently crosslinked by a covalent crosslinker, wherein the covalent crosslinker is a bis-acrylamide compound or a bis-acrylate compound.
Aspect 85. The copolymer of Aspect 84, wherein the covalent crosslinker is selected from the group consisting of methylenebisacrylamide, polyethyleneglycol diacrylate, ethylene glycol dimethacrylate, N-(1-hydroxy-2,2-dimethoxyethyl)acrylamide, divinylbenzene, phenylenediacrylamide, diurethane dimethacrylate, and any combination thereof.
Aspect 86. The copolymer of Aspect 84 or 85, wherein the covalent crosslinker is in the amount greater than 0 mole % to about 35 mole percent.
Aspect 87. The copolymer of any one of Aspects 76-86, wherein the residue from Group (a) is from about 40 mole percent to about 90 mole percent of the polymer.
Aspect 88. The copolymer of any one of Aspects 76-87, wherein Group (b) is greater than 0 mole % to about 40 mole percent of the polymer.
Aspect 89. The copolymer of any one of Aspects 76-88, wherein the residue from Group (c) is greater than 0 mole % to about 35 mole percent of the polymer.
Aspect 90. The copolymer of any one of Aspects 76-89, wherein the polymer has an average molecular weight from about 1,000 Da to about 1,000,000 Da.
Aspect 91. A copolymer comprising at least one residue from the monomers in Group (a) and at least one residue from the monomers in Group (b):
   a. acrylamide, methacrylamide, an alkyl acrylamide, a dialkyl acrylamide, an alkyl methacrylamide, a dialkyl methacrylamide, or any combination thereof; and
   b. one or more alkyl acrylates,
   wherein the polymer is covalently crosslinked.
Aspect 92. A copolymer comprising at least one residue from the monomers in Group (a) and at least one residue from the monomers in Group (b):
   a. acrylamide, methacrylamide, an alkyl acrylamide, a dialkyl acrylamide, an alkyl methacrylamide, a dialkyl methacrylamide, or any combination thereof; and
   b. one or more alkyl acrylates,
   wherein the polymer is not a copolymer of n-isopropylacrylamide/acrylic acid, n-isopropylacrylamide/methacrylic acid, n-isopropylacrylamide/ethyl acrylate, n-isopropylacrylamide/butyl acrylate, n-isopropylacrylamide/lauryl acrylate, n-isopropylacrylamide/stearyl acrylate, n-isopropylacrylamide/methacrylic acid, n-isopropylacrylamide/, n-isopropylacrylamide/, n-isopropylacrylamide/, n-isopropylacrylamide/methyl methacrylate, n-isopropylacrylamide/iso-octyl acrylate, n-isopropylacrylamide/dimethylaminoethyl methacrylate, acrylamide/acrylic acid, acrylamide/methacrylic acid, or acrylamide/alkyl ester acrylates.
Aspect 93. A nail polish system wherein the primary solvent comprises ethanol.
Aspect 94. The nail polish of Aspect 93, wherein the nail polish system comprises a primary film former selected from the group consisting of a cellulose ester, an acrylic resin, polyester, an alkyd, or a vinyl polymer.
Aspect 95. The nail polish of Aspect 93, wherein the nail polish system comprises a primary film former selected from the group consisting of cellulose acetate butyrate, acrylic polymer, vinyl acetate/butyl maleate/IBA, imidized isobutylene/maleic anhydride copolymer, polyvinyl methyl ether/maleic acid copolymer, or a polymer containing an alkyl acrylamide, a dialkyl acrylamide, an alkyl methacrylamide, or a dialkyl methacrylamide.
Aspect 96. A method for decorating a substrate, the method comprising applying the coating composition of any one of Aspects 1-35 in a specified pattern or design to at least one surface of the substrate to produce a decorated substrate.
Aspect 97. The method of Aspect 96, wherein the substrate is a body part of a subject.
Aspect 98. The method of Aspect 96, wherein the substrate is a fingernail or toenail of a subject.
Aspect 99. The method of any one of Aspects 96-98, wherein prior to applying the coating composition to the substrate, the substrate was previously coated with a composition that is not the coating composition of any one of Aspects 1-35.
Aspect 100. A coating composition comprising a solvent and a copolymer comprising at least one residue from the monomers in Group (a) and at least one residue from the monomers in Group (b):
   a. acrylamide, methacrylamide, an alkyl acrylamide, a dialkyl acrylamide, an alkyl methacrylamide, a dialkyl methacrylamide, or any combination thereof; and
   b. at least one C₁-C₁₈ alkyl acrylate.
Aspect 101. The coating composition of Aspect 100, wherein the Group (a) monomer is selected from the group consisting of n-methyl acrylamide, n-ethylacrylamide, n-isopropylacrylamide, n-butylacrylamide, n-tertbutylacrylamide, n-methylmethacrylamide, n-ethyl methacrylamide, n-isopropyl methacrylamide, n-butyl methacrylamide, n-tertbutyl methacrylamide, and any combination thereof.
Aspect 102. The coating composition of Aspect 100, wherein the Group (a) monomer is an alkyl methacrylamide.
Aspect 103. The coating composition of Aspect 100, wherein the Group (a) monomer is n-isopropyl methacrylamide.
Aspect 104. The coating composition of Aspect 100, wherein the C₁-C₁₈ alkyl acrylate is selected from the group consisting of methyl acrylate, ethyl acrylate, propyl acrylate, butyl acrylate, pentyl acrylate, hexyl acrylate, 2-ethylhexyl acrylate, benzyl acrylate, heptyl acrylate, octyl acrylate, nonyl acrylate, decyl acrylate, undecyl acrylate, dodecyl acrylate, hexadecyl acrylate, stearyl acrylate, lauryl acrylate, t-butyl acrylate, isobutyl acrylate, isobornyl acrylate and any combination thereof.
Aspect 105. The coating composition of Aspect 100, wherein the copolymer comprises at least two C₁-C₁₈ alkyl acrylates.
Aspect 106. The coating composition of Aspect 100, wherein the C₁-C₁₈ alkyl acrylate comprises ethyl hexyl acrylate and a second alkyl acrylate.
Aspect 107. The coating composition of any one of Aspects 100-106, wherein the copolymer further comprises a residue from acrylic acid, 2-acrylamido-2-methyl-1-propanesulfonic acid, 4-styrenesulfonic acid tetrabutylammonium salt, methyl methacrylate, vinyl acetate, methacrylic acid, ethyl maleate, isopropyl maleate, butyl maleate, isobornyl acrylate, isobornyl methacrylate, styrene, n-isopropylmethacrylamide, and dimethyl aminoethyl methacrylate, or any combination thereof.
Aspect 108. The coating composition of Aspect 100, wherein the copolymer comprises a residue of an alkyl methacrylamide and at least two C₁-C₁₈ alkyl acrylates.
Aspect 109. The coating composition of Aspect 100, wherein the copolymer comprises residues from n-isopropylmethacrylamide, dodecyl acrylate, and ethylhexyl acrylate.
Aspect 110. The coating composition of any one of Aspects 100-109, wherein the copolymer is covalently crosslinked with a crosslinker.
Aspect 111. The coating composition of Aspect 110, wherein the crosslinker is a bis-acrylamide compound or a bis-acrylate compound.
Aspect 112. The coating composition of Aspect 110, wherein the covalent crosslinker is selected from the group consisting of methylenebisacrylamide, polyethyleneglycol diacrylate, ethylene glycol dimethacrylate, N-(1-hydroxy-2,2-dimethoxyethyl)acrylamide, divinylbenzene, phenylenediacrylamide, diurethane dimethacrylate, and any combination thereof.
Aspect 113. The coating composition of any one of Aspects 100-112, wherein the residue from Group (a) is from about 40 mole percent to about 90 mole percent of the polymer and the residue from Group (b) is from about 0.1 mole percent to about 40 mole percent of the polymer.
Aspect 114. The coating composition of any one of Aspects 100-113, wherein the copolymer further comprises a residue from acrylic acid, methyl methacrylate, methacrylic acid, and dimethyl aminoethyl methacrylate, or any combination thereof in the amount from about 0.1 mole percent to about 35 mole percent.
Aspect 115. The coating composition of any one of Aspects 100-114, wherein the copolymer has an average molecular weight from about 1,000 Da to about 1,000,000 Da.
Aspect 116. The coating composition of any one of Aspects 100-115, wherein the solvent comprises an alcohol.
Aspect 117. The coating composition of any one of Aspects 100-115, wherein the solvent comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, water, propylene glycol, glycerin, propylene carbonate, dimethyl glutarate, dimethyl adipate, methyl soyate, 2,2,4-trimethyl-1,3-pentanediol monoisobutyrate, denatured alcohol, diacetone alcohol, 1-methoxy, dimethoxy propanol, ethoxy propanol, or any combination thereof.
Aspect 118. The coating composition of any one of Aspects 100-117, wherein the coating composition further comprises a secondary polymer comprising a polyester, an alkyd, a polyurethane, a tosylamide formaldehyde resin, an acrylic resin, vinyl acetate/butyl maleate/IBA copolymer, imidized isobutylene/maleic anhydride copolymer, polyvinyl methyl ether/maleic acid copolymer, a cellulose ester, a rosinate, adipic acid/neopentyl glycol/trimellitic anhydride copolymer, hexanedioic acid, cellulose acetate butyrate, nitrocellulose, cellulose acetate propionate, cellulose acetate phthalate, hydroxypropyl cellulose, hydroxyethyl cellulose, a polycondensate of adipic acid/(esterified reaction product of glycerin and coconut oil fatty acid)/2,2′-(41ethylamino)diethanol, tridecyl trimellitate, or any combination thereof.
Aspect 119. The coating composition of any one of Aspects 100-118, wherein the coating composition further comprises a filler, wherein the filler is selected from the group consisting of hectorite, stearalkonium hectorite, bentonite, bentonite, stearalkonium bentonite, silica, glass microspheres, hydrophobic fumed silica, hydrophilic fumed silica, silica dimethyl silylate, organophilic phyllosilicate, kaolin clay, halloysite, montmorillonite, barium sulfate, salt, or any combination thereof.
Aspect 120. The coating composition of any one of Aspects 100-118, wherein the coating composition further comprises a pigment.
Aspect 121. The coating of Aspect 120, wherein the pigment comprises a dispersion.
Aspect 122. The coating composition of any one of Aspects 100-121, wherein the coating composition further comprises a plasticizer, a humectant, a dispersant, a suspension agent, a lipid, an oil, a vitamin, a biological molecule, an essential oil, a plant extract, a plant hydrosol, a fragrance, a preservative, a thickener, one or more color additives, a pH adjuster, a surfactant, or any combination thereof.
Aspect 123. A protective coating composition comprising a polymer and solvent, wherein the solvent comprises an alcohol.
Aspect 124. The protective coating composition of Aspect 123, wherein the alcohol comprises ethanol or denatured alcohol.
Aspect 125. The protective coating composition of Aspect 123 or 124, wherein the polymer is selected from a cellulose ester or an alkoxylated cellulose.
Aspect 126. The protective coating composition of any one of Aspects 123-125, wherein the composition comprises an alkyl methacrylamide monomer.
Aspect 127. The protective coating composition of any one of Aspects 123-126, wherein the composition comprises a primary film former selected from the group consisting of cellulose acetate butyrate, acrylic polymer, vinyl acetate/butyl maleate/IBA, imidized isobutylene/maleic anhydride copolymer, polyvinyl methyl ether/maleic acid copolymer, or a polymer containing an alkyl acrylamide, a dialkyl acrylamide, an alkyl methacrylamide, or a dialkyl methacrylamide.
Aspect 128. A coating removal solution comprising a solvent, wherein the solvent comprises an alcohol and a second solvent, wherein the mass of the alcohol is greater than the mass of the second solvent, and the solution removes a coating composition on a subject.
Aspect 129. The removal solution of Aspect 128, wherein the second solvent comprises water, propylene carbonate, or a combination thereof.
Aspect 130. The removal solution of Aspect 128, wherein the removal solution comprises ethanol and water.
Aspect 131. A kit comprising the coating composition of any one of Aspects 100-122 and an applicator.
Aspect 132. The kit of Aspect 131, wherein the kit further comprises a protective coating composition.
Aspect 133. The kit of Aspect 131 or 132, wherein the protective coating comprises a cellulose ester, cellulose acetate butyrate, cellulose acetate propionate, cellulose acetate phthalate, ethyl cellulose, alkoxylated cellulose, or nitrocellulose.
Aspect 134. The kit of any one of Aspects 131-133, wherein the kit further comprises a removal composition comprising an alcohol.
Aspect 135. The kit of Aspect 134, wherein the removal solution further comprises a vitamin, protein, polysaccharide, salt, peptide, plant extract, oil, hydrosol, a humectant, an exfoliant, a dispersant, a suspension agent, a preservative, a thickener, an acid, a base, an enzyme, a filler, a color additive, a pH adjuster, a surfactant, a salt, or any combination thereof.
Aspect 136. A method for coating a substrate, the method comprising applying the coating composition of any one of Aspects 100-122 to at least one surface of the substrate to produce a coated substrate.
Aspect 137. The method of Aspect 136, wherein the substrate is a body part of a subject.
Aspect 138. The method of Aspect 136 or 137, wherein the coating composition is applied by brushing, dipping, or spray coating the coating composition on the substrate.
Aspect 139. The method of any one of Aspects 136-138, wherein the method further comprises applying a protective coating composition to the coated substrate.
Aspect 140. A copolymer comprising at least one residue from the monomers in Group (a), and at least one residue from the monomers in Group (b):
   a. methacrylamide, an alkyl methacrylamide, a dialkyl methacrylamide, or any combination thereof; and
   b. one or more C₁ to C₁₈ alkyl acrylates.
Aspect 141. The copolymer of Aspect 140, wherein the group (a) residue is n-isopropyl methacrylamide.
Aspect 142. The copolymer of Aspect 140 or 141, wherein one group (b) residue is ethylhexyl acrylate.
Aspect 143. The copolymer of any one of Aspects 140-142, wherein the polymer is covalently crosslinked with a crosslinker.
Aspect 144. The copolymer of Aspect 143, wherein the crosslinker is selected from the group consisting of methylenebisacrylamide, polyethyleneglycol diacrylate, ethylene glycol dimethacrylate, N-(1-hydroxy-2,2-dimethoxyethyl)acrylamide, divinylbenzene, phenylenediacrylamide, diurethane dimethacrylate, and any combination thereof.
Aspect 145. The copolymer of any one of Aspects 140-144, wherein the residue from Group (a) is from about 40 mole percent to about 90 mole percent of the polymer and the residue from Group (b) is greater than 0 mole percent to about 40 mole percent of the polymer.
Aspect 146. The copolymer of any one of Aspects 140-145, wherein the polymer has an average molecular weight from about 1,000 Da to about 1,000,000 Da.
Aspect 147. A copolymer comprising at least one residue from the monomers in Group (a) and at least one residue from the monomers in Group (b):
   a. acrylamide, methacrylamide, an alkyl acrylamide, a dialkyl acrylamide, an alkyl methacrylamide, a dialkyl methacrylamide, or any combination thereof; and
   b. one or more C₁-C₁₈ alkyl acrylates,
   wherein the polymer is covalently crosslinked.
Aspect 148. A copolymer comprising at least one residue from the monomers in Group (a) and at least one residue from the monomers in Group (b):
   a. acrylamide, methacrylamide, an alkyl acrylamide, a dialkyl acrylamide, an alkyl methacrylamide, a dialkyl methacrylamide, or any combination thereof; and
   b. one or more C₁-C₁₈ alkyl acrylates,
   wherein the polymer is not a copolymer of n-isopropylacrylamide/acrylic acid, n-isopropylacrylamide/methacrylic acid, n-isopropylacrylamide/ethyl acrylate, n-isopropylacrylamide/butyl acrylate, n-isopropylacrylamide/lauryl acrylate, n-isopropylacrylamide/stearyl acrylate, n-isopropylacrylamide/methacrylic acid, n-isopropylacrylamide/, n-isopropylacrylamide/, n-isopropylacrylamide/, n-isopropylacrylamide/methyl methacrylate, n-isopropylacrylamide/iso-octyl acrylate, n-isopropylacrylamide/dimethylaminoethyl methacrylate, acrylamide/acrylic acid, acrylamide/methacrylic acid, or acrylamide/alkyl ester acrylates.
Aspect 149. A coating composition comprising a solvent and a copolymer comprising at least one residue from the monomers in Group (a) and at least one residue from the monomers in Group (b):
   a. acrylamide, methacrylamide, an alkyl acrylamide, a dialkyl acrylamide, an alkyl methacrylamide, a dialkyl methacrylamide, or any combination thereof; and
   b. at least one C₁-C₁₈ alkyl acrylate.
Aspect 150. The coating composition of Aspect 149, wherein the Group (a) monomer is selected from the group consisting of n-methyl acrylamide, n-ethylacrylamide, n-isopropylacrylamide, n-butylacrylamide, n-tertbutylacrylamide, n-methylmethacrylamide, n-ethyl methacrylamide, n-isopropyl methacrylamide, n-butyl methacrylamide, n-tertbutyl methacrylamide, and any combination thereof.
Aspect 151. The coating composition of Aspect 149, wherein the Group (a) monomer is an alkyl methacrylamide.
Aspect 152. The coating composition of Aspect 149, wherein the Group (a) monomer is n-isopropyl methacrylamide.
Aspect 153. The coating composition of Aspect 149, wherein the C₁-C₁₈ alkyl acrylate is selected from the group consisting of methyl acrylate, ethyl acrylate, propyl acrylate, butyl acrylate, pentyl acrylate, hexyl acrylate, 2-ethylhexyl acrylate, benzyl acrylate, heptyl acrylate, octyl acrylate, nonyl acrylate, decyl acrylate, undecyl acrylate, dodecyl acrylate, hexadecyl acrylate, stearyl acrylate, lauryl acrylate, t-butyl acrylate, isobutyl acrylate, isobornyl acrylate and any combination thereof.
Aspect 154. The coating composition of Aspect 149, wherein the copolymer comprises at least two C₁-C₁₈ alkyl acrylates.
Aspect 155. The coating composition of Aspect 149, wherein the C₁-C₁₈ alkyl acrylate comprises ethyl hexyl acrylate and a second alkyl acrylate.
Aspect 156. The coating composition of any one of Aspects 149-155, wherein the copolymer further comprises a residue from acrylic acid, 2-acrylamido-2-methyl-1-propanesulfonic acid, 4-styrenesulfonic acid tetrabutylammonium salt, methyl methacrylate, vinyl acetate, methacrylic acid, ethyl maleate, isopropyl maleate, butyl maleate, isobornyl acrylate, isobornyl methacrylate, styrene, n-isopropylmethacrylamide, and dimethyl aminoethyl methacrylate, or any combination thereof.
Aspect 157. The coating composition of Aspect 149, wherein the copolymer comprises a residue of an alkyl methacrylamide and at least two C₁-C₁₈ alkyl acrylates.
Aspect 158. The coating composition of Aspect 149, wherein the copolymer comprises residues from n-isopropylmethacrylamide, dodecyl acrylate, and ethylhexyl acrylate.
Aspect 159. The coating composition of any one of Aspects 149-158, wherein the copolymer is covalently crosslinked with a crosslinker.
Aspect 160. The coating composition of Aspect 159, wherein the crosslinker is a bis-acrylamide compound or a bis-acrylate compound.
Aspect 161. The coating composition of Aspect 159, wherein the covalent crosslinker is selected from the group consisting of methylenebisacrylamide, polyethyleneglycol diacrylate, ethylene glycol dimethacrylate, N-(1-hydroxy-2,2-dimethoxyethyl)acrylamide, divinylbenzene, phenylenediacrylamide, diurethane dimethacrylate, and any combination thereof.
Aspect 162. The coating composition of any one of Aspects 149-161, wherein the residue from Group (a) is from about 40 mole percent to about 90 mole percent of the polymer and the residue from Group (b) is from about 0.1 mole percent to about 40 mole percent of the polymer.
Aspect 163. The coating composition of any one of Aspects 149-162, wherein the copolymer further comprises a residue from acrylic acid, methyl methacrylate, methacrylic acid, and dimethyl aminoethyl methacrylate, or any combination thereof in the amount from about 0.1 mole percent to about 35 mole percent.
Aspect 164. The coating composition of any one of Aspects 149-163, wherein the copolymer has an average molecular weight from about 1,000 Da to about 1,000,000 Da.
Aspect 165. The coating composition of any one of Aspects 149-164, wherein the solvent comprises an alcohol.
Aspect 166. The coating composition of any one of Aspects 149-164, wherein the solvent comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, water, propylene glycol, glycerin, propylene carbonate, dimethyl glutarate, dimethyl adipate, methyl soyate, 2,2,4-trimethyl-1,3-pentanediol monoisobutyrate, denatured alcohol, diacetone alcohol, 1-methoxy, dimethoxy propanol, ethoxy propanol, or any combination thereof.
Aspect 167. The coating composition of any one of Aspects 149-166, wherein the coating composition further comprises a secondary polymer comprising a polyester, an alkyd, a polyurethane, a tosylamide formaldehyde resin, an acrylic resin, vinyl acetate/butyl maleate/IBA copolymer, imidized isobutylene/maleic anhydride copolymer, polyvinyl methyl ether/maleic acid copolymer, a cellulose ester, a rosinate, adipic acid/neopentyl glycol/trimellitic anhydride copolymer, hexanedioic acid, cellulose acetate butyrate, nitrocellulose, cellulose acetate propionate, cellulose acetate phthalate, hydroxypropyl cellulose, hydroxyethyl cellulose, a polycondensate of adipic acid/(esterified reaction product of glycerin and coconut oil fatty acid)/2,2′-(47ethylamino)diethanol, tridecyl trimellitate, or any combination thereof.
Aspect 168. The coating composition of any one of Aspects 149-167, wherein the coating composition further comprises a filler, wherein the filler is selected from the group consisting of hectorite, stearalkonium hectorite, bentonite, bentonite, stearalkonium bentonite, silica, glass microspheres, hydrophobic fumed silica, hydrophilic fumed silica, silica dimethyl silylate, organophilic phyllosilicate, kaolin clay, halloysite, montmorillonite, barium sulfate, salt, or any combination thereof.
Aspect 169. The coating composition of any one of Aspects 149-167, wherein the coating composition further comprises a pigment.
Aspect 170. The coating of Aspect 69, wherein the pigment comprises a dispersion.
Aspect 171. The coating composition of any one of Aspects 149-170, wherein the coating composition further comprises a plasticizer, a humectant, a dispersant, a suspension agent, a lipid, an oil, a vitamin, a biological molecule, an essential oil, a plant extract, a plant hydrosol, a fragrance, a preservative, a thickener, one or more color additives, a pH adjuster, a surfactant, or any combination thereof.
Aspect 172. A protective coating composition comprising a polymer and solvent, wherein the solvent comprises an alcohol.
Aspect 173. The protective coating composition of Aspect 172, wherein the polymer comprises a cellulose ester, cellulose acetate butyrate, cellulose acetate propionate, cellulose acetate phthalate, ethyl cellulose, alkoxylated cellulose, nitrocellulose, or any combination thereof.
Aspect 174. A removal composition comprising a solvent, wherein the solvent comprises an alcohol.
Aspect 175. A kit comprising the coating composition of any one of Aspects 149-171 and an applicator.
Aspect 176. The kit of Aspect 175, wherein the kit further comprises a protective coating composition.
Aspect 177. The kit of Aspect 175 or 176, wherein the kit further comprises a removal composition comprising an alcohol.
Aspect 178. A method for coating a substrate, the method comprising applying the coating composition of any one of Aspects 149-171 to at least one surface of the substrate to produce a coated substrate.
Aspect 179. The method of Aspect 179, wherein the substrate is a body part of a subject.
Aspect 180. The method of Aspect 178 or 179, wherein the method further comprises applying a protective coating composition to the coated substrate.

### EXAMPLES

The following examples are put forth so as to provide those of ordinary skill in the art with a complete disclosure and description of how the compounds, compositions, and methods described and claimed herein are made and evaluated and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, the temperature is in ° C. or is at ambient temperature, and pressure is at or near atmospheric. Numerous variations and combinations of reaction conditions (e.g., component concentrations, desired solvents, solvent mixtures, temperatures, pressures, and other reaction ranges and conditions) can be used to further optimize the reagent consumption while at the same time increase the extraction efficiency.

### Example 1—Synthesis of a Vinyl Copolymer by FRP

NIPAM (70 g), butyl acrylate (20 g), Acrylic Acid (10 g), and AIBN (0.322 g) were dissolved in 160 mL of denatured alcohol. The solution was purged with nitrogen for 2 hours and then heated to 55C for 4 hours under inert atmosphere. The reaction mixture was allowed to cool and then precipitated into water with agitation to remove impurities. Product is then either redissolved into an organic solvent for spray drying or dried in a vacuum to remove residue before grinding to a powder.

### Example 2—Synthesis of a Vinyl Copolymer by FRP

n-isopropylmethacrylate (70 g), butyl acrylate (20 g), Acrylic Acid (10 g), and AIBN (0.322 g) were dissolved in 160 mL of denatured alcohol. The solution was purged with nitrogen for 2 hours and then heated to 55C for 4 hours under inert atmosphere. The reaction mixture was allowed to cool and then precipitated into water with agitation to remove impurities. Product is then either redissolved into an organic solvent for spray drying or dried in a vacuum to remove residue before grinding to a powder.

### Example 3—Synthesis of a Vinyl Copolymer by FRP

NIPAM (79.95 g), ethylhexyl acrylate (20 g), methylene biscrylamide (0.05 g), and AIBN (0.322 g) were dissolved in 160 mL of denatured alcohol. The solution was purged with nitrogen for 2 hours and then heated to 55C for 4 hours under inert atmosphere. The reaction mixture was allowed to cool and then precipitated into water with agitation to remove impurities. Product is then either redissolved into an organic solvent for spray drying or dried in a vacuum to remove residue before grinding to a powder.

### Example 4—Synthesis of a Vinyl Copolymer by FRP

N-isopropylmethacrylamide (69.95 g), ethylhexyl acrylate (20 g), Dodecyl acrylate (10 g), polyethylene glycol diacrylate (0.05 g), and AIBN (0.322 g) were dissolved in 160 mL of denatured alcohol. The solution was purged with nitrogen for 2 hours and then heated to 55C for 8 to 24 hours under inert atmosphere. The reaction mixture was then either used as-is or was allowed to cool and then precipitated into water with agitation to remove impurities. If product was precipitated, it was then either redissolved into an organic solvent for spray drying or dried in a vacuum to remove residue before grinding to a powder.

### Example 5—Synthesis of Alternate Vinyl Copolymer by FRP

NIPAM (69.95 g), ethylhexyl acrylate (20 g), Acrylic Acid (10 g), methylene biscrylamide (0.05 g), and AIBN (0.322 g) were dissolved in 160 mL of denatured alcohol. The solution was purged with nitrogen for 2 hours and then heated to 55C for 4 hours under inert atmosphere. The reaction mixture was allowed to cool and then precipitated into water with agitation to remove impurities. Product is then either redissolved into an organic solvent for spray drying or dried in a vacuum to remove residue before grinding to a powder.

### Example 6—Synthesis of Alternate Vinyl Copolymer by FRP

NIPAM (69.95 g), ethylhexyl acrylate (20 g), methyl methacrylate (10 g), methylene biscrylamide (0.05 g), and AIBN (0.322 g) were dissolved in 160 mL of denatured alcohol. The solution was purged with nitrogen for 2 hours and then heated to 55C for 4 hours under inert atmosphere. The reaction mixture was allowed to cool and then precipitated into water with agitation to remove impurities. Product is then either redissolved into an organic solvent for spray drying or dried in a vacuum to remove residue before grinding to a powder.

### Example 7—Synthesis of Alternate Vinyl Copolymer by RAFT

NIPAM (80 g), ethylhexyl acrylate (40 g), methylene biscrylamide (0.1 g), 2-(dodecylthiocarbonothioylthio)-2-methylpropionic acid (1 g), and AIBN (0.5 g) were dissolved in 200 mL of denatured alcohol. The solution was purged with nitrogen for 2 hours with stirring and then heated to 55C for 4 hours under inert atmosphere. The reaction mixture was allowed to cool and then precipitated into water with agitation to remove impurities. Product is then either redissolved into an organic solvent for spray drying or dried in a vacuum to remove residue before grinding to a powder.

### Example 8—Synthesis of Alternate Vinyl Copolymer by RAFT

NIPAM (80 g), ethylhexyl acrylate (40 g), methylene biscrylamide (0.1 g), CuBr (0.05 g), PMDETA (0.05 g), and BriB (0.05 g) were dissolved in 200 ml of denatured alcohol. The solution was purged with nitrogen for 2 hours and then heated to 55C for 4 hours under inert atmosphere. The reaction mixture was allowed to cool and then precipitated into water with agitation to remove impurities. Product is then either redissolved into an organic solvent for spray drying or dried in a vacuum to remove residue before grinding to a powder.

| Example 9-Vinyl Polymer Base Coating Formula (w/W) | |
|---|---|
| Ethanol or denatured alcohol | 69% |
| Vinyl Polymer | 25% |
| Polyester Resin | 6% |

| Example 10-Vinyl Polymer Base Coating Formula (w/W) | |
|---|---|
| Ethanol or denatured alcohol | 69% |
| Vinyl Polymer | 25% |
| Polyester Resin | 5% |
| Acrylic Resin | 1% |

| Example 11-Vinyl Polymer Base Coating Formula (w/W) | |
|---|---|
| Ethanol or denatured alcohol | 68.9% |
| Vinyl Polymer | 22% |
| Polyester Resin | 6% |
| Silica | 2% |
| Niacinamide | 0.5% |
| Panthenol | 0.5% |
| Centella Asiatica Extract | 0.1% |

| Example 12-Vinyl Polymer Color Coating Formula (w/W) | |
|---|---|
| Ethanol or denatured alcohol | 60% |
| Vinyl Polymer | 25% |
| Sucrose acetate isobutyrate | 4% |
| Pigment | 2% |
| Fumed Silica | 1.5% |
| Polyester Resin | 1.5% |
| Triethyl Acetyl Citrate | 4.5% |
| Stearalkonium Bentonite | 0.5% |
| Hydroxyacetophenone | 0.5% |
| Surfactant | 0.5% |

| Example 13-Vinyl Polymer Therapeutic Color Coating Formula (w/W) | |
|---|---|
| Ethanol or denatured alcohol | 60% |
| Vinyl Polymer | 30% |
| Pigment | 2% |
| Fumed Silica | 1.5% |
| Tributyl Acetyl Citrate | 3.5% |
| Stearalkonium Bentonite | 0.5% |
| Hydroxyacetophenone or | 0.5% |
| other UV protectant | |
| Surfactant | 0.5% |
| Glycerin | 0.5% |
| Vitamins, Plant Extracts | 1% |

| Example 14-Protective Cellulose Ester Coating (w/W) | |
|---|---|
| Ethanol or denatured alcohol | 65.5% |
| Diacetone Alcohol | 11% |
| Cellulose acetate butyrate | 18% |
| Polyester Resin | 3% |
| Polyvinyl butyral | 1.5% |
| Acrylic Resin | 1% |

| Example 15-Polar Coating Removal Solution (w/W) | |
|---|---|
| Ethanol | 50% |
| Water | 50% |
| Denaturants | <0.1% |

| Example 16-Polar Coating Removal Solution (w/W) | |
|---|---|
| Ethanol | 50% |
| Water | 30% |
| Propylene Carbonate | 20% |
| Denaturants | <0.1% |

| Example 17-Therapeutic Polar Coating Removal Solution (w/W) | |
|---|---|
| Ethanol | 50% |
| Water | 28% |
| Propylene Carbonate | 20% |
| Glycerin | 1% |
| Vitamins, Plant Extracts | 1% |
| Denaturants | <0.1% |

| Example 18-Therapeutic Hydrosol Polar Coating Removal Solution (w/W) | |
|---|---|
| Ethanol | 50% |
| Rose Water | 28% |
| Propylene Carbonate | 20% |
| Glycerin | 1% |
| Vitamins, Plant Extracts | 1% |
| Denaturants | <0.1% |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A coating composition comprising a solvent and a copolymer comprising at least one residue from the monomers in Group (a) and at least one residue from the monomers in Group (b):
a. acrylamide, methacrylamide, an alkyl acrylamide, a dialkyl acrylamide, an alkyl methacrylamide, a dialkyl methacrylamide, or any combination thereof; and
b. at least one C₁-C₁₈ alkyl acrylate.

2. The coating composition of claim 1, wherein the Group (a) monomer is selected from the group consisting of n-methyl acrylamide, n-ethylacrylamide, n-isopropylacrylamide, n-butylacrylamide, n-tertbutylacrylamide, n-methylmethacrylamide, n-ethyl methacrylamide, n-isopropyl methacrylamide, n-butyl methacrylamide, n-tertbutyl methacrylamide, and any combination thereof.

3. The coating composition of claim 1, wherein the Group (a) monomer is an alkyl methacrylamide.

4. The coating composition of claim 1, wherein the Group (a) monomer is n-isopropyl methacrylamide.

5. The coating composition of claim 1, wherein the C₁-C₁₈ alkyl acrylate is selected from the group consisting of methyl acrylate, ethyl acrylate, propyl acrylate, butyl acrylate, pentyl acrylate, hexyl acrylate, 2-ethylhexyl acrylate, benzyl acrylate, heptyl acrylate, octyl acrylate, nonyl acrylate, decyl acrylate, undecyl acrylate, dodecyl acrylate, hexadecyl acrylate, stearyl acrylate, lauryl acrylate, t-butyl acrylate, isobutyl acrylate, isobornyl acrylate and any combination thereof.

6. The coating composition of claim 1, wherein the copolymer comprises at least two C₁-C₁₈ alkyl acrylates.

7. The coating composition of claim 1, wherein the C₁-C₁₈ alkyl acrylate comprises ethyl hexyl acrylate and a second alkyl acrylate.

8. The coating composition of claim 1, wherein the copolymer further comprises a residue from acrylic acid, 2-acrylamido-2-methyl-1-propanesulfonic acid, 4-styrenesulfonic acid tetrabutylammonium salt, methyl methacrylate, vinyl acetate, methacrylic acid, ethyl maleate, isopropyl maleate, butyl maleate, isobornyl acrylate, isobornyl methacrylate, styrene, n-isopropylmethacrylamide, and dimethyl aminoethyl methacrylate, or any combination thereof.

9. The coating composition of claim 1, wherein the copolymer comprises a residue of an alkyl methacrylamide and at least two C₁-C₁₈ alkyl acrylates.

10. The coating composition of claim 1, wherein the copolymer comprises residues from n-isopropylmethacrylamide, dodecyl acrylate, and ethylhexyl acrylate.

11. The coating composition of claim 1, wherein the copolymer is covalently crosslinked with a crosslinker.

12. The coating composition of claim 11, wherein the crosslinker is a bis-acrylamide compound or a bis-acrylate compound.

13. The coating composition of claim 11, wherein the covalent crosslinker is selected from the group consisting of methylenebisacrylamide, polyethyleneglycol diacrylate, ethylene glycol dimethacrylate, N-(1-hydroxy-2,2-dimethoxyethyl)acrylamide, divinylbenzene, phenylenediacrylamide, diurethane dimethacrylate, and any combination thereof.

14. The coating composition of claim 1, wherein the residue from Group (a) is from about 40 mole percent to about 90 mole percent of the polymer and the residue from Group (b) is from about 0.1 mole percent to about 40 mole percent of the polymer.

15. The coating composition of claim 1, wherein the copolymer further comprises a residue from acrylic acid, methyl methacrylate, methacrylic acid, and dimethyl aminoethyl methacrylate, or any combination thereof in the amount from about 0.1 mole percent to about 35 mole percent.

16. The coating composition of claim 1, wherein the copolymer has an average molecular weight from about 1,000 Da to about 1,000,000 Da.

17. The coating composition of claim 1, wherein the solvent comprises an alcohol.

18. The coating composition of claim 1, wherein the solvent comprises ethyl alcohol, methyl alcohol, isopropanol, sec-butanol, n-butanol, iso-butanol, acetone, ethyl acetate, butyl acetate, 1-methoxy-2 propanol, diacetone alcohol, anisole, propyl acetate, toluene, xylene, methyl ethyl ketone, water, propylene glycol, glycerin, propylene carbonate, dimethyl glutarate, dimethyl adipate, methyl soyate, 2,2,4-trimethyl-1,3-pentanediol monoisobutyrate, denatured alcohol, diacetone alcohol, 1-methoxy, dimethoxy propanol, ethoxy propanol, or any combination thereof.

19. The coating composition of claim 1, wherein the coating composition further comprises a secondary polymer comprising a polyester, an alkyd, a polyurethane, a tosylamide formaldehyde resin, an acrylic resin, vinyl acetate/butyl maleate/IBA copolymer, imidized isobutylene/maleic anhydride copolymer, polyvinyl methyl ether/maleic acid copolymer, a cellulose ester, a rosinate, adipic acid/neopentyl glycol/trimellitic anhydride copolymer, hexanedioic acid, cellulose acetate butyrate, nitrocellulose, cellulose acetate propionate, cellulose acetate phthalate, hydroxypropyl cellulose, hydroxyethyl cellulose, a polycondensate of adipic acid/(esterified reaction product of glycerin and coconut oil fatty acid)/2,2′-(3ethylamino)diethanol, tridecyl trimellitate, or any combination thereof.

20. The coating composition of claim 1, wherein the coating composition further comprises a filler, wherein the filler is selected from the group consisting of hectorite, stearalkonium hectorite, bentonite, bentonite, stearalkonium bentonite, silica, glass microspheres, hydrophobic fumed silica, hydrophilic fumed silica, silica dimethyl silylate, organophilic phyllosilicate, kaolin clay, halloysite, montmorillonite, barium sulfate, salt, or any combination thereof.

21. The coating composition of claim 1, wherein the coating composition further comprises a pigment.

22. The coating of claim 21, wherein the pigment comprises a dispersion.

23. The coating composition of claim 1, wherein the coating composition further comprises a plasticizer, a humectant, a dispersant, a suspension agent, a lipid, an oil, a vitamin, a biological molecule, an essential oil, a plant extract, a plant hydrosol, a fragrance, a preservative, a thickener, one or more color additives, a pH adjuster, a surfactant, or any combination thereof.

24. A protective coating composition comprising a polymer and solvent, wherein the solvent comprises an alcohol.

25. The protective coating composition of claim 24, wherein the polymer comprises a cellulose ester, cellulose acetate butyrate, cellulose acetate propionate, cellulose acetate phthalate, ethyl cellulose, alkoxylated cellulose, nitrocellulose, or any combination thereof.

26. A removal composition comprising a solvent, wherein the solvent comprises an alcohol.

27. A kit comprising the coating composition of claim 1 and an applicator.

28. The kit of claim 27, wherein the kit further comprises a protective coating composition.

29. The kit of claim 27, wherein the kit further comprises a removal composition comprising an alcohol.

30. A method for coating a substrate, the method comprising applying the coating composition of claim 1 to at least one surface of the substrate to produce a coated substrate.

31. The method of claim 30, wherein the substrate is a body part of a subject.

32. The method of claim 30, wherein the method further comprises applying a protective coating composition to the coated substrate.
